(19) United States
(12) Patent Application Publication
(10) Pub. No.: US 2025/0009243 A1
(43) Pub. Date: Jan. 9, 2025

(54) METHOD AND DEVICE FOR AUTOMATED CHARACTERISATION OF OESOPHAGEAL CATHETERS HAVING A BALLOON PROBE

(71) Applicant: Hamilton Medical AG, Bonaduz (CH)

(72) Inventor: David Schwenninger, Felsberg (CH)

(73) Assignee: Hamilton Medical AG, Bonaduz (CH)

(21) Appl. No.: 18/705,799

(22) PCT Filed: Oct. 18, 2022
(86) PCT No.: PCT/EP2022/078954

§ 371 Date: Apr. 29, 2024

(30) Foreign Application Priority Data
Oct. 29, 2021 (DE) 10 2021 128 271.3

(51) Int. Cl. A61B 5/03; A61B 5/00; A61M 16/00; A61M 25/10

(52) U.S. Cl. CPC A61B 5/037; A61B 5/6853; A61M 16/0003; A61B 2562/0247; A61B 2562/08; A61M 2016/0027; A61M 25/10187

(57) ABSTRACT

A characterization system for automated ex vivo characterization of an esophageal catheter with a balloon probe for determining an esophageal pressure comprises an arrangement for filling the esophageal catheter with a measuring fluid before placing the esophageal catheter in an esophagus, a pressure sensor for detecting a pressure (pₑₛₒ) prevailing in the esophageal catheter; a device for determining at least one property that is characteristic of the esophageal catheter on the basis of the pressure (pₑₛₒ) detected in the esophageal catheter; a storage device for storing at least the property characteristic of the esophageal catheter; and at least one controller for controlling the characterization system, the controller being designed such that the characterization system performs a method comprising at least the following steps: filling the esophageal catheter with a measuring fluid ex vivo, in particular before placing the esophageal catheter in an esophagus; detecting a pressure prevailing in the esophageal catheter; determining at least one property characteristic of the esophageal catheter on the basis of the detected pressure; and storing at least the property characteristic of the esophageal catheter.

### BACKGROUND

The present invention relates to a method and a device for automated characterization of esophageal catheters with balloon probes.

Esophageal balloon catheters with balloon probes for determining an esophageal balloon pressure are used in particular in mechanical ventilation in order to determine the transpulmonary pressure in the chest of a patient.

In today's common forms of mechanical ventilation, the patient is supplied with breathing gas at a positive pressure. Therefore, during ventilation, the airway pressure or alveolar pressure is greater than the pressure in the pleural space or gap surrounding the pulmonary alveoli, at least during the inspiration phase. During the expiration phase, there is no pressurization of the airway by the ventilation device, with the result that the lung tissue relaxes and the airway pressure or alveolar pressure decreases. Under certain circumstances, this kind of positive pressure ventilation can cause the pressure conditions in the airway or in the alveoli to become so unfavorable at the end of the expiration phase that parts of the alveoli collapse. The collapsed part of the lung volume then must first be unfolded again in the subsequent breathing cycle. The functional residual capacity of the lungs is severely impaired, so that oxygen saturation decreases, and the lung tissue also suffers permanent damage.

To prevent collapse of alveoli at the end of the expiration phase, a so-called positive end-expiratory pressure, commonly briefly referred to as PEEP, is usually set during positive pressure mechanical ventilation. With this measure, an improvement in oxygen saturation can be achieved in many cases.

When ventilating with PEEP, the ventilation device applies a predetermined positive pressure, the PEEP, to the airway permanently—i.e. both during the inspiration phase and during the expiration phase. The PEEP is therefore still present after the end of the expiration phase.

Ideally, the PEEP should be set sufficiently high so that during the expiration phase the alveolar pressure is not, or at least only to such an extent, below the pressure in the pleural gap that the alveolar tissue does not collapse under the effect of the pressure in the pleural gap. In other words, the PEEP is to prevent the transpulmonary pressure-which is the pressure difference between alveolar pressure and pressure in the pleural gap—from becoming lower than zero or from falling below a lower negative limit value at which parts of the alveoli begin to collapse.

On the other hand, a too high value of the PEEP may have a negative effect, especially during the inspiration phase. This is because the lung tissue can become overstretched at very high airway pressures during the inspiration phase. Numerous studies also indicate that a high value of the PEEP can impede the return flow of venous blood to the heart with corresponding negative effects on the cardiovascular system.

In itself, the PEEP should be matched to the respective prevailing transpulmonary pressure. However, the transpulmonary pressure in a ventilated patient is not amenable to simple determination. To get by, one therefore measures the pressure in a balloon probe placed in the esophagus of a ventilated patient by means of an esophageal balloon catheter. With suitable positioning and configuration of the balloon, the pressure measured in the balloon can be used to approximately determine the pressure in the pleural gap.

The document WO 2014/037175 A1 describes the automated setting of a pressure predefined by a ventilation device, in particular the positive end-expiratory pressure (PEEP) and the maximum airway pressure, on the basis of the esophageal balloon pressure which is regarded as an indicator of the transpulmonary pressure, i.e. the pressure difference between alveolar pressure and pressure in the pleural gap.

In practice, the relationship between the pressure measured in a balloon catheter inserted into the esophagus and the pressure in the pleural gap may change during ventilation. The causes for this can be manifold and generally cannot be determined in detail.

Mojoli et al, Crit. Care (2016) 20:98 and Hotz et al, Respir. Care (2018) 63 (2): 177-186 recommend procedures for calibrating an esophageal balloon catheter with a balloon probe, which is intended for measuring the esophageal pressure. The aim of the calibration is to achieve optimum filling of the balloon probe with air, in which the balloon probe is as sensitive as possible to changes in the pressure in the pleural gap acting on the esophagus and reflects the pressure in the pleural gap as well as possible.

However, the measurement procedures described by Mojoli et al. for calibration are complex and fragile, so that in practice only an optimum filling for the balloon probe can be determined and preset before the start of ventilation. This optimum filling is then maintained during ventilation and is not changed further.

### SUMMARY

It is an object of the invention to improve the accuracy of pressure measurement values determined using esophageal catheters after they have been inserted into a patient's esophagus. It is also desirable to accelerate and simplify the use of esophageal catheters in mechanical ventilation and to reduce the risk of incorrect use of an esophageal catheter, which can lead to incorrect ventilation.

The invention comprises a method for automated characterization of an esophageal catheter with a balloon probe, which is provided for determining an esophageal pressure. A method according to the invention comprises the following steps:
filling the esophageal catheter with a measuring fluid ex vivo, i.e. without the esophageal catheter having been placed in an esophagus;
detecting a pressure prevailing in the esophageal catheter;
determining at least one property characteristic of the esophageal catheter on the basis of the detected pressure; and
storing the at least one property characteristic of the balloon probe.

The measuring fluid which is filled into the esophageal catheter can be air or a different ventilation gas, e.g. oxygen-enriched air.

It is a basic idea of the invention to prepare esophageal catheters for use in mechanical ventilation of patients by means of a ventilation device, in particular to determine characteristic properties of the esophageal catheters prior to their use in mechanical ventilation.

The characteristic property that is determined can be, in particular, a characteristic parameter of the esophageal catheter that is associated with a specific esophageal balloon pressure and that is detected and stored in order to be used at a later time, in particular when controlling automated ventilation of a patient.

The characteristic parameter may comprise, for example, a characteristic quantity of measuring fluid in the esophageal catheter or a characteristic volume of the esophageal catheter corresponding to this characteristic quantity.

In addition to the characteristic quantity of measuring fluid in the esophageal catheter or the characteristic volume corresponding to this characteristic quantity, the characteristic parameter may also comprise a detected balloon pressure that is associated with the characteristic quantity. For example, an upper filling quantity N_{full} of measuring fluid in the esophageal catheter at a predetermined maximum pressure p_{full} can be defined as the quantity of measuring fluid in the esophageal catheter at a predetermined balloon pressure of e.g. 15 hPa.

The characteristic volume of the esophageal catheter is determined on the basis of the general gas equation from the characteristic quantity of measuring fluid in the esophageal catheter and the measured balloon pressure.

With a dynamic determination of the characteristic parameter, i.e. with a mass flow of the measuring fluid into or out of the esophageal catheter which is greater than zero or in any case significantly different from zero, it can be advantageous to compensate the respectively measured pressure by a ram pressure of the fluid flow which results from the flow resistance of the esophageal catheter.

The invention also comprises a system (“characterization system”) for automated ex vivo characterization of an esophageal catheter with a balloon probe which can be inserted into the esophagus of a patient to be ventilated and which is provided for determining an esophageal pressure, wherein the characterization system comprises
an arrangement for filling the esophageal catheter with a measuring fluid ex vivo, i.e. before the esophageal catheter is placed in an esophagus;
a pressure sensor for detecting a pressure prevailing in the esophageal catheter;
a device for determining at least one property characteristic of the esophageal catheter on the basis of the pressure detected in the esophageal catheter;
a storage device for storing the at least one property characteristic of the esophageal catheter; and
a controller for controlling the system, which is adapted to control the system to carry a method according to the invention for automated characterization of an esophageal catheter with a balloon probe.

The invention also comprises a ventilation device comprising a system according to the invention for automated characterization of an esophageal catheter that can be inserted into the esophagus of a patient to be ventilated.

The invention further comprises an esophageal catheter with a balloon probe for insertion into the esophagus of a patient to be ventilated, which is provided for determining an esophageal pressure, in particular during mechanical ventilation by means of a ventilation device. The esophageal catheter comprises a balloon probe which can be inserted into the esophagus of a patient to be ventilated and to which a measuring fluid can be applied, and a fluid interface for connection to a system according to the invention. The fluid interface is designed such that the system is capable of determining at least one property characteristic of the esophageal catheter via the fluid interface.

The characterization of an esophageal catheter that can be inserted into the esophagus of a patient to be ventilated, according to the invention, makes it possible to improve the accuracy of pressure measurement values provided by the esophageal catheter after it has been inserted into the esophagus of a patient, in particular the reproduction of the pleural pressure prevailing in the pleural gap of the patient. The invention therefore renders possible better and more efficient use of esophageal catheters in the control of automated ventilation of patients.

In the case of esophageal catheters, a distinction must be made between the volume and the amount of fluid (“fluid quantity”) in the esophageal catheter. The fluid quantity refers to the number of molecules or the molar quantity of the fluid.

The ideal gas equation describes the relationship between the volume V and the fluid quantity N at a predefined pressure p and at a given temperature T:

pV=N kT,
wherein the following applies:
p=pressure of the fluid
V=volume of the fluid
N=number of molecules in the fluid
k=Boltzmann constant, and
T=temperature of the fluid.

In the context of the present invention, the following applies in particular: The temperature does virtually not change when carrying out the methods according to the invention and can therefore be regarded as a constant factor.

The quantity N of measuring fluid in the esophageal catheter when a certain pressure p₁ is reached is determined by time integration over a fluid flow measured by a flow sensor between a time to, at which the initial pressure p₀ was set, and a later time t₁, at which a predetermined pressure p₁ is reached.

Apart from a temperature-dependent factor kT, which is constant at constant temperature, the flow sensor thus measures the expression on the right-hand side of the ideal gas equation (N kT).

The respective pressure pₑₛₒ prevailing in the esophageal catheter is also measured, for example, by a pressure sensor arranged at the distal end of the esophageal catheter, as positive pressure pₑₛₒ with respect to the ambient pressure p_{amb}. Consequently, the pressure p on the left-hand side of the ideal gas equation represents the sum of the ambient pressure p_{amb} and the positive pressure pₑₛₒ in the esophageal catheter: p=(p_{amb}+pₑₛₒ).

This means that instead of the variable N kT, which apart from the temperature-dependent factor kT, which is constant here, represents the quantity of measuring fluid in the balloon probe, it is also possible to specify the volume V=N kT/(p_{amb}+pₑₛₒ) that is currently occupied by the entire system of the esophageal catheter.

This volume V is made up of the “system volume” V_{sys} of the supply lines (and other components, if applicable) and the volume V_{b} of the balloon probe of the esophageal catheter. If the tubes (and possibly other components present) are considered rigid with respect to the pressures occurring during the methods or processes described herein, the system volume V_{sys} is a constant. The volume of the balloon probe V_{b}, on the other hand, changes when measuring fluid is added to or withdrawn from the balloon probe.

In this regard, the following relationship holds:

$$( {p}_{amb} + {p}_{eso} ) ( {V}_{sys} + {V}_{b} ) = N kT = kT \int q ( t ) d t ,$$

when q(t) is the fluid flow into and out of the esophageal catheter, respectively.

With regard to the positive pressure pₑₛₒ, it may be necessary to note that the ram pressure, which is a result of the flow resistance R of the esophageal catheter, must be subtracted from the measured pressure in order to obtain pₑₛₒ.

The flow resistance R of an esophageal catheter is another characteristic parameter of the esophageal catheter. A method for determining the flow resistance R of the esophageal catheter is part of the present invention and will be described further below.

Using the relationship shown above, an associated volume V_{b} of the balloon probe can be calculated for each measurement value of the pressure pₑₛₒ if V_{sys} can be neglected with respect to V_{b} or if the system volume V_{sys} is known and can thus be taken into account in the calculation.

The system volume V_{sys} is also a characteristic parameter of an esophageal catheter that can be determined using a method as described further below.

If the balloon probe has virtually no elastance and therefore expands or contracts in such a way that the total volume V=V_{sys}+V_{b} of the esophageal catheter changes in proportion to the quantity N of measuring fluid added or withdrawn, the pressure pₑₛₒ in the balloon probe and in the esophageal catheter remains constant when measuring fluid is added to or withdrawn from the esophageal catheter.

A curve describing the relationship between the pressure pₑₛₒ and the quantity N of measuring fluid in the balloon probe therefore has an essentially linear range which, when the pressure pₑₛₒ is plotted on the y axis above the fluid quantity N on the x axis, ideally runs horizontally, i.e. parallel to the x axis. However, if elastance is present, the linear part of the curve increases with increasing balloon pressure p_{B}.

In the context of the description of the invention, all variables derived from the integral of the fluid flow q(t) are based on the measuring fluid quantity N in the balloon probe or in the esophageal catheter and not on the volume occupied by the fluid.

The fluid quantity N is used instead of the volume V in order to express that the fluid quantity N in the balloon probe or esophageal catheter is concerned at which a predetermined pressure pₑₛₒ is measured. This fluid quantity N can be converted into a volume V_{b} of the balloon probe by multiplying the measured fluid quantity N by the current temperature T, dividing the same by the pressure (p_{amb}+pₑₛₒ) and then subtracting the system volume V_{sys}:

$${V}_{b} = {N}_{b} kT / ( {p}_{amb} + peso ) - {V}_{sys} .$$

In the following, optional implementations of the invention will be described with reference to possible embodiments. It is to be understood that the features mentioned in each case for one embodiment are optional and may be combined with the features of other embodiments described herein, unless this is expressly excluded.

In one embodiment, the invention comprises setting a filling quantity, i.e. a quantity of measuring fluid in the esophageal catheter, and recording the time course of the pressure and the filling quantity in the esophageal catheter in order to then determine at least one characteristic property of the esophageal catheter on the basis of the recorded pressure course and the recorded course of the filling quantity.

When recording the course of the pressure and the filling quantity, it is in particular possible to proceed in such a way that several pairs of measurement values are recorded, wherein each pair of measurement values comprises a detected pressure (first measurement value of the pair) and a filling quantity associated with the respective pressure (second measurement value of the pair). The filling quantity can be a relative filling quantity, which has been determined as a change in the filling quantity compared to a predetermined start value or a previously determined filling quantity.

In one embodiment, in order to determine the at least one property characteristic of the esophageal catheter, it may be provided to change the filling quantity between a start value and an end value of a predetermined measurement range that is predefined for determining the characteristic property of the esophageal catheter, and to determine the change in the filling quantity with respect to a predefined start value or a previously determined filling quantity at each of the measurement points resulting in this way and, in addition, to record a pressure in the esophageal catheter detected in each case by a pressure sensor and to associate said pressure with the respective filling quantity.

In one embodiment, it may be provided to determine a measurement range for the balloon probe in accordance with the detected balloon pressure in order to determine the at least one property characteristic of the balloon probe. The measurement range, over which important information for characterizing the esophageal catheter is obtained, can in particular comprise a range from −30 hPa (negative pressure) to +28 hPa (positive pressure) with respect to the ambient pressure p_{amb}. The measurement range may also comprise one or more partial ranges of the aforementioned range.

A separate measurement range can be associated with a respective characteristic property or a group of respective characteristic properties. For example, a measurement range between −30 hPa and −10 hPa can be associated with the determination of the system volume V_{sys} of the esophageal catheter. The determination of characteristic properties/parameters N_{full}, Nₘₐₓ and Nₘᵢₙ, which characterize the linear range of the esophageal catheter described above, may have a common measurement range associated therewith, for example a measurement range covering a range from −5 hPa to +15 hPa. The meaning of the parameters N_{full}, Nₘₐₓ and Nₘᵢₙ will be described in detail later.

Additional measurement ranges, which may differ from the aforementioned measurement ranges, can be associated with the determination of a leakage rate and/or the determination of the flow resistance R_{flow} of the esophageal catheter.

The measurement ranges mentioned can also overlap in partial ranges or be identical.

In one embodiment, a start value or an end value of a range used to determine at least one characteristic property may correspond to a quantity of measuring fluid in the balloon probe that is greater than an upper limit of the respective measurement range provided for the esophageal catheter.

In one embodiment, a start value or an end value of a range used to determine at least one characteristic property may correspond to a quantity of measuring fluid in the balloon probe that is less than a lower limit of the respective measurement range provided for the esophageal catheter.

In one embodiment, in a method for determining the characteristic parameters N_{full}, Nₘₐₓ and/or Nₘᵢₙ, the upper limit of the measurement range can be the quantity N=N_{full} of measuring fluid at which the pressure pₑₛₒ=p_{full} is reached in the esophageal catheter. The start value N_{full} of the measurement for determining the limits Nₘₐₓ and Nₘᵢₙ of the linear range is preferably selected even higher than the upper limit Nₘₐₓ, so that a certain prestretching of the balloon probe of the esophageal catheter occurs.

In one embodiment, it is possible to determine a plurality of measurement values for the pressure in the esophageal catheter for a respectively set filling quantity or for a respective measurement point between the start value and the end value. An average and a statistical variance for the measurement value or a variable derived therefrom are then determined on the basis of the plurality of measurement values. In this way, the accuracy and reliability of the method can be improved.

In particular, the step size between successive measurement points can be determined in adaptive manner. Such an adaptive determination of the step size can be carried out, for example, by way of a gradient method, such as the Newton algorithm.

In one embodiment, the characteristic property or parameter determined by a method according to the invention may comprise one or more of an upper (or maximum) filling quantity N_{full} of the esophageal catheter, a filling quantity of the esophageal catheter corresponding to a filling quantity upper limit Nₘₐₓ of a linear range of the esophageal catheter, a filling quantity of the esophageal catheter corresponding to a filling quantity lower limit Nₘᵢₙ of a linear range, a nominal filling quantity of the esophageal catheter, a system volume V_{sys} of the esophageal catheter, a flow resistance R of the esophageal catheter and a leakage rate of the esophageal catheter. The characteristic property may also be an n-tuple whose components are n selected properties of the aforementioned properties of the esophageal catheter.

An esophageal catheter can be characterized and, if necessary, classified by each of the aforementioned properties or by a combination of two of the more of the aforementioned properties, in particular by an n-tuple comprising several of the aforementioned properties.

In one embodiment, the method for determining a characteristic property of the esophageal catheter may comprise a method for detecting a leakage in the esophageal catheter.

In one embodiment, a method for detecting a leakage in the esophageal catheter may comprise, in a first step, bringing or inflating the esophageal catheter and in particular the balloon probe of the esophageal catheter to a first positive pressure pₒ₁, for example by means of the pumping device. The esophageal catheter can, for example, be brought to a first positive pressure pₒ₁ of at least +8 hPa, in particular to a first positive pressure pₒ₁ of +15 hPa, with respect to the ambient pressure p_{amb}.

The method may comprise furthermore waiting a predetermined waiting time t_{wait} in a second step to allow complete pressure equalization within the esophageal catheter including the balloon probe. The predetermined waiting time t_{wait} can be between 1 s and 10 s, for example. In particular, the predetermined waiting time t_{wait} can be between 2 s and 5 s, for example about 3 s.

After the predetermined waiting time t_{wait} has elapsed, the pressure pₑₛₒ in the esophageal catheter can be reduced to a second positive pressure pₒ₂, which is less than the first positive pressure pₒ₁ (pₒ₂<pₒ₁).

In the subsequent step, the pressure pₒ₃ in the esophageal catheter is measured over a predetermined period of time Δt without introducing further measuring fluid into the esophageal catheter or withdrawing measuring fluid from the esophageal catheter during this period of time Δt. The predetermined period of time Δt may comprise, for example, periods of time Δt in the range from 5 s to 15 s, in particular a period of about 10 s.

If the pressure drop Δp=pₒ₂−pₒ₃ in the esophageal catheter measured within the predetermined period of time Δt exceeds a predefined limit value Δpₗᵢₘᵢₜ (Δp>Δpₗᵢₘᵢₜ), a message is issued indicating that the system consisting of esophageal catheter and characterization system is leaking. This can be due in particular to a defective esophageal catheter, but also due to a leaking connection between the esophageal catheter and the characterization system.

If the pressure drop Δp=pₒ₂−pₒ₃ in the esophageal catheter measured within the predetermined period of time Δt does not exceed the predefined limit value Δpₗᵢₘᵢₜ (Δp≤Δpₗᵢₘᵢₜ), it is assumed that the esophageal catheter meets a standard applicable to leakage and can be used to monitor and control the ventilation of a patient (“in vivo”) from this point of view. Therefore, a corresponding message is issued. Optionally, the esophageal catheter can also be marked accordingly.

Setting the first positive pressure pₒ₁ in preparation for the actual leakage measurement is optional. This means that in an alternative method, the setting of a first pressure pₒ₁ before the actual measurement can be dispensed with. In this case, the predetermined period of time Δt over which the pressure drop in the esophageal catheter is observed is generally longer than in the method described above, in which a first positive pressure pₒ₁>pₒ₂ is set in the esophageal catheter in preparation for the actual leakage measurement.

Furthermore, especially if the esophageal catheter has been assessed as usable, the previously measured pressure drop Δp or the previously measured pressure drop rate Δp/Δt can also be stored as a characteristic property or characteristic parameter of the esophageal catheter for subsequent use.

If the pressure drop rate Δp/Δt of an esophageal catheter is known, this can be taken into account during subsequent use of the esophageal catheter in a patient (“in vivo”) and, if necessary, compensated for by calculation or by filling the esophageal catheter with measuring fluid. In addition, the esophageal catheter can be easily and reliably recognized as defective during subsequent operation if a pressure drop rate Δp/Δt determined during use on the patient exceeds the pressure drop rate Δp/Δt previously determined as a characteristic parameter of the esophageal catheter by more than a predetermined tolerance.

A method for detecting leakage in the esophageal catheter can also be performed with negative pressure, instead of positive pressure as described above. In this case, instead of the positive pressures pₒ₁, pₒ₂, pₒ₃ mentioned above, different negative pressures pᵤ₁, pᵤ₂, pᵤ₃ are successively set in the esophageal catheter, i.e. pressures pᵤ₁, pᵤ₂, pᵤ₃ that are lower than the ambient pressure p_{amb}. The negative pressure in the esophageal catheter is reduced step by step, i.e. it is brought closer to the ambient pressure p_{amb} “from below”.

A method with positive pressure and a method with negative pressures can also be carried out one after the other, and the two methods can be carried out in arbitrary order. The volumes introduced into and withdrawn from the esophageal catheter while performing each method can be measured and compared with each other. A difference between these volumes can be used to draw conclusions about a leakage in the system comprising the esophageal catheter and the characterization system.

In one embodiment, the characteristic property or characteristic parameter of the esophageal catheter can be the system volume V_{sys} of the esophageal catheter. The system volume V_{sys} of an esophageal catheter is defined as the volume of the esophageal catheter that is present in the esophageal catheter when the balloon probe is completely collapsed so that its volume is “zero”.

The system volume V_{sys} is essentially determined by the volume of the catheter tube of the esophageal catheter. The system volume V_{sys} may also comprise the volumes of additional elements of the esophageal catheter that are in fluid connection with the catheter tube, for example fluid couplings, connectors, etc. The catheter tube and the additional elements are usually so rigid that their volumes do not change with the pressure changes occurring during operation and characterization of the esophageal catheter or change so little compared to the volume of the balloon probe that these changes in volume are negligible. Therefore, the system volume V_{sys} determined at p=p_{amb} can be regarded as a constant. The system volume V_{sys} can therefore be regarded as a “real” volume which is given, for example, in cm³ or milliliters, and not as a fluid quantity which is given, for example, in mol.

In one embodiment, a system according to the invention may comprise a pumping device which is designed to introduce measuring fluid into the balloon probe and/or withdraw measuring fluid from the balloon probe in accordance with control commands from the controller. In particular, the pumping device may comprise a fluid pump and/or a valve that can be controlled by the controller. With the help of such a pumping device, the quantity of measuring fluid into the balloon probe can be controlled and/or regulated and set to values predefined by the controller.

In one embodiment, the controller can be designed to control and/or regulate the mass flow of measuring fluid introduced into and/or withdrawn from the balloon probe. With such a controller, the mass flow of the measuring fluid introduced into the balloon probe and/or withdrawn from the balloon probe can be reliably set to a predetermined value.

In one embodiment, a system according to the invention may also comprise a sensor which is designed to detect a mass flow of measuring fluid introduced into the balloon probe and/or withdrawn from the balloon probe. With the aid of such a sensor, the mass flow of measuring fluid introduced into the balloon probe and/or withdrawn from the balloon probe can be reliably determined.

In one embodiment of a method for determining the system volume V_{sys}, the esophageal catheter can be evacuated in a first step, e.g. with the aid of a pumping device. For example, the esophageal catheter can be evacuated until a first negative pressure pₑₛₒ=pᵤ₁ has been set in the esophageal catheter. The first negative pressure pᵤ₁ can be −30 hPa, for example. The pressure specifications given here and in the following always refer to the ambient pressure p_{amb}, i.e. a negative pressure pᵤ₁ of −30 hPa means that the pressure pₑₛₒ in the esophageal catheter is 30 hPa below the ambient pressure p_{amb}.

In a second step, the negative pressure pₑₛₒ in the esophageal catheter can be reduced by introducing fluid into the esophageal catheter, i.e. the pressure in the esophageal catheter is increased by introducing fluid into the esophageal catheter.

For example, a second negative pressure pₑₛₒ=pᵤ₂ is set in this way. The second negative pressure pᵤ₂ can be −20 hPa, for example. In order to reduce the negative pressure in the esophageal catheter by introducing fluid into the esophageal catheter, fluid that is under a higher pressure than pᵤ₂ can be allowed to flow into the esophageal catheter by opening the controllable valve. Alternatively or additionally, fluid can also be actively pumped into the esophageal catheter by the fluid pump.

After a predetermined waiting time t_{wait} has elapsed, which may be a few seconds, the negative pressure in the esophageal catheter can be reduced still further by introducing additional fluid into the esophageal catheter. For example, enough fluid can be introduced into the esophageal catheter to set a third negative pressure pₑₛₒ=pᵤ₃. The third negative pressure pᵤ₃ can be −10 hPa, for example.

All three negative pressures pᵤ₁, pᵤ₂, pᵤ₃ mentioned should be so far below the ambient pressure p_{amb} that the balloon probe of the esophageal catheter is always completely collapsed and does not contribute anything to the volume of the esophageal catheter.

While the negative pressure in the esophageal catheter, as described above, is reduced from the second negative pressure pᵤ₂ to the third negative pressure pᵤ₃, i.e. while the pressure pₑₛₒ in the esophageal catheter is increased from the second negative pressure pᵤ₂ to the third negative pressure pᵤ₃, the pressure course p(t) in the esophageal catheter and the mass flow q(t) of the measuring fluid flowing into the esophageal catheter are measured.

If a flow sensor used for this purpose also detects the pressure pₑₛₒ currently prevailing in the esophageal catheter, or if the pressure pₑₛₒ in the esophageal catheter is known from another measurement, a volume flow v(t) corresponding to the mass flow q(t) can also be detected by the flow sensor instead of the mass flow q(t) and, when the temperature is known, can be used to determine the system volume V_{sys} and other characteristic properties of the esophageal catheter.

The pressure dependency of the relationship between mass flow and volume flow can be negligible in the context of the pressure changes applied here relative to the required accuracy. In this case, a volume flow v(t) measured by the flow sensor can also be used directly to determine the system volume V_{sys} and other characteristic properties of the esophageal catheter without determining the associated mass flow.

The fluid quantity ΔN (additionally) introduced into the esophageal catheter results from an integration of the mass flow q(t) measured by the flow sensor over the time t between a starting point pₑₛₒ=pᵤ₂ and an end point pₑₛₒ=pᵤ₃:

ΔN=∫q(t)dt.

Using the general gas equation, the system volume V_{sys} can be determined from the measured pressure difference Δp=pᵤ₃−pᵤ₂, the fluid quantity introduced into the esophageal catheter ΔN and the current temperature T as a characteristic property of the esophageal catheter and stored for subsequent use.

The method described here for determining the system volume V_{sys} presupposes that the system volume V_{sys} remains constant during the measurement, i.e. during the change in the (negative) pressure pₑₛₒ in the esophageal catheter between the start point pᵤ₂ and the end point pᵤ₃. This means that the balloon probe of the esophageal catheter should not be deployed during any of the measurements, as otherwise its volume would change and the additional volume would contribute to the total volume of the esophageal catheter.

The condition of a completely collapsed balloon probe is in any case fulfilled if the pressures pᵤ₂ and pᵤ₃ are significantly below the ambient pressure p_{amb}, as described above. Moreover, it is assumed that the additional components of the esophageal catheter, i.e. all components apart from the balloon itself, especially the lines, are sufficiently rigid at the (negative) pressures pᵤ₂ and pᵤ₃ used here, so that their flexibility is negligible.

If the system volume V_{sys} of an esophageal catheter is known, the quantity of measuring fluid N_{sys} can be determined in advance that is necessary, starting from a state in which a predetermined negative pressure pₑₛₒ=pᵤ₂ (e.g. pᵤ₂=−20 hPa) is measured in the esophageal catheter, to fill the esophageal catheter to such an extent that pressure equalization with the ambient pressure p_{amb} takes place, so that the pressure pₑₛₒ measured in the esophageal catheter relative to the ambient pressure p_{amb} would be zero, using the ideal gas equation:

$${N}_{s y s} = ( ( {p}_{amb} - {p}_{eso} ) {V}_{sys} ) / ( k T ) ,$$

wherein k is Boltzmann's constant and T is the temperature.

Knowing the system volume V_{sys} and the fluid quantity N_{sys} makes it easier to calibrate the esophageal catheter in vivo, i.e. in a patient's esophagus, because there is no need to wait for the pressure pₑₛₒ in the esophageal catheter to equalize with the ambient pressure p_{amb}. Rather, it is sufficient to subtract the calculated fluid quantity N_{sys} from the respective determined quantity of measuring fluid in the esophageal catheter.

In one embodiment, a method according to the invention may comprise determining the flow resistance R_{flow} as a characteristic property/characteristic parameter of an esophageal catheter.

For determining the flow resistance R_{flow} of an esophageal catheter, a predetermined filling level of the esophageal catheter can be set in a first step. The predetermined filling level can be specified by a predefined fluid quantity No, a predefined filling volume V₀, or by a predefined pressure p₀ in the esophageal catheter.

After the predetermined filling level in the esophageal catheter has been set, the fluid quantity in the esophageal catheter can be changed in a subsequent step, in particular by the pumping device, in pulse-like manner, i.e. with a relatively large time gradient. For this purpose, additional measuring fluid is introduced into the esophageal catheter or withdrawn from the esophageal catheter within a sufficiently short period of time, so that the pressure in the esophageal catheter changes rapidly, i.e. with a large time gradient.

For example, the fluid quantity in the esophageal catheter can be changed with a gradient of 3.6*10−3 mol/min (or 90 ml/min).

The period of time in which the fluid quantity in the esophageal catheter is changed can have a duration in the range of 0.2 s to 1 s.

At the same time, i.e. while the fluid quantity in the esophageal catheter is changed in pulse-like manner with a large time gradient as described above, the mass flow q(t) and the pressure p(t) in the esophageal catheter are measured, in particular with the aid of the flow sensor described above.

From the mass flow q(t) measured in this way and the pressure p(t) measured in the esophageal catheter, the temporal changes dq of the flow rate q(t) and the temporal change dp of the pressure p(t) are determined.

The flow resistance R_{flow}=dp/dq of the esophageal catheter then results as the quotient of the previously determined pressure change dp and the previously determined temporal change dq of the mass flow. The flow resistance R_{flow} determined in this way is then stored as a characteristic property or characteristic parameter of the esophageal catheter for subsequent use.

Alternatively, R_{flow} can also be calculated by solving the equation

$$P ( t ) = {R}_{Flow} * q ( t ) + X * V ( t ) + P 0$$

using linear regression for the variables R_{Flow}, X and P₀.

In one embodiment, the method for determining a characteristic property of the esophageal catheter may comprise determining a filling quantity upper limit Nₘₐₓ and a filling quantity lower limit Nₘᵢₙ of a linear range such that there is a substantially linear relationship between the pressure p and the filling quantity N within the esophageal catheter when the filling quantity N of the esophageal catheter changes in the range between the filling quantity lower limit Nₘᵢₙ and the filling quantity upper limit Nₘₐₓ (Nₘᵢₙ<N<Nₘₐₓ).

To determine the filling quantity upper limit Nₘₐₓ and the filling quantity lower limit Nₘᵢₙ of the linear range, the esophageal catheter is first filled with measuring fluid, e.g. using the pumping device, until a specified upper pressure p_{full} (pₑₛₒ=p_{full}) is reached inside the esophageal catheter. The specified upper pressure p_{full} corresponds to an upper filling quantity N_{full} or an upper filling volume V_{full} of the esophageal catheter. The upper filling volume V_{full} of the esophageal catheter results from the upper filling quantity N_{full} and the temperature T according to the general gas equation pV=N kT mentioned above.

The upper pressure p_{full} and the upper filling quantity N_{full} are selected such that the upper filling quantity N_{full} is above the expected filling quantity upper limit Nₘₐₓ of the linear range. When the esophageal catheter has been brought to the predefined upper pressure p_{full} and the corresponding upper filling quantity N_{full}, the balloon probe of the esophageal catheter is expanded and filled with more measuring fluid than intended for operation, the balloon probe is therefore overstretched. However, the overstretching is not so severe that there is a risk of the balloon probe being damaged by excessive fluid pressure.

Since p_{full} in vivo, i.e when the esophageal catheter is inserted into the esophagus of a patient, cannot be measured directly due to the pressure then exerted on the esophageal catheter by the esophagus, setting of the upper filling quantity N_{full} or of the upper filling volume V_{full} before the esophageal catheter is inserted into the esophagus of a patient permits faster, more targeted and safer overfilling of the balloon catheter prior to refilling and/or renewed calibration of the esophageal catheter than if this quantity is not known. Without knowledge of the upper filling quantity N_{full} or the upper filling volume V_{full}, safe overfilling of the esophageal catheter in vivo is significantly more difficult and involves higher risks. In particular, if the upper filling quantity N_{full} and the upper filling volume V_{full} are not known, there is a considerable risk that the esophageal catheter expands to a too large volume or with a too high pressure and is damaged by overstretching.

In one embodiment, in order to determine N_{full}, and if desired Nₘₐₓ and/or Nₘᵢₙ, it may be provided to fill the balloon probe in a first measurement cycle to such an extent that a positive pressure is measured which is slightly greater than the pressure p_{full} associated with N_{full}, e.g. a positive pressure of 15 hPa. The quantity of measuring fluid introduced into the esophageal catheter is measured using a mass flow sensor as described above.

Starting from this start value, measuring fluid can then be drained and/or pumped out of the esophageal catheter in a second measurement cycle until the pressure pₑₛₒ in the esophageal catheter has dropped to the ambient pressure p_{amb} or even to a pressure below the ambient pressure p_{amb}, e.g. to a negative pressure of −5 hPa. The quantity ΔN of the measuring fluid withdrawn from the esophageal catheter can be measured with the mass flow sensor. N_{full} can then be determined as the quantity of measuring fluid in the esophageal catheter at which the predetermined positive pressure p_{full} is measured in the second measurement cycle.

Optionally, N_{full} can be converted into a volume using the equation

$${V}_{full} = ( {N}_{full} kT ) / ( {p}_{amb} + {p}_{full} ) - Vsys$$

wherein N_{full} is given in mol, k is the Boltzmann constant and T is the temperature.

The predetermined upper pressure p_{full} can be in a range from 10 hPa to 20 hPa, for example, in particular the predetermined upper pressure p_{full} can be a pressure of about 15 hPa above the ambient pressure p_{amb}.

In the method described above for determining a respective filling quantity N_{full} of measuring fluid to be introduced into or withdrawn from the esophageal catheter until a respective predetermined pressure p with respect to the ambient pressure p_{amb} is reached, it can be taken into account that in the case of dynamic measurement, i.e. in a measurement in which the mass flow q of the fluid is greater than zero, the respective measured pressure in the esophageal catheter must be applied in part as ram pressure pᵣₐₘ to overcome a flow resistance R of the esophageal catheter. This applies, for example, when setting the upper filling pressure p_{full}.

In order to compensate for this ram pressure pᵣₐₘ, which is dependent on the mass flow q of the measuring fluid conveyed from the esophageal catheter during the introduction or discharge of measuring fluid, it is possible to determine the flow resistance R_{Flow} which the measuring fluid flow through the esophageal catheter must overcome, and to compensate the respective measurement values for the pressure in the esophageal catheter by the ram pressure pᵣₐₘ in order to obtain the pressure pₑₛₒ in the balloon probe, assuming that the pressure measurement takes place outside the esophageal catheter. The ram pressure pᵣₐₘ is the product of the flow resistance R_{Flow} and the mass flow q of the measuring fluid flowing out of the esophageal catheter:

−pᵣₐₘ=q R_{Flow}.

If the mass flow q of the measuring fluid remains small during introduction into the esophageal catheter or during discharge from the esophageal catheter, the ram pressure compensation caused by the flow resistance R_{Flow} can be negligible. In this case, the quantity of measuring fluid in the esophageal catheter associated with the respective pressure in the esophageal catheter results directly as the quantity of measuring fluid AN that has been introduced into the esophageal catheter or withdrawn from the esophageal catheter starting from a start value until a pressure p corresponding to a respective measurement point is measured in the esophageal catheter.

If the mass flow q is so large that the ram pressure pᵣₐₘ must be taken into account, the quantity of measuring fluid in the esophageal catheter associated with the predetermined pressure in the esophageal catheter, after this has been converted into a volume, is calculated on the basis of the predetermined compensated positive pressure p_{system} as follows:

Nₘₑₐₛ=N_{b}(P_{system}),
wherein p_{system}=a pressure corresponding to the respective measurement point with

$${p}_{system} = {p}_{meas} - {p}_{ram} = {p}_{meas} - {R}_{Flow} q ,$$

wherein R_{Flow} is the flow resistance and q is the mass flow.

After the pressure pₑₛₒ in the esophageal catheter has been brought to the predetermined upper pressure p_{full}, the pressure pₑₛₒ in the esophageal catheter is gradually reduced by withdrawing fluid, in particular with the aid of the pumping device. For this purpose, fluid can be drained from the esophageal catheter by opening a valve of the pumping device. Alternatively or additionally, the fluid can also be actively pumped out of the esophageal catheter by a fluid pump.

The fluid quantity N in the esophageal catheter can be reduced starting from the upper filling quantity N_{full}, in particular to such an extent that a negative pressure, for example a negative pressure of −5 hPa, is set in the esophageal catheter with respect to the ambient pressure p_{amb}.

The measuring fluid can be withdrawn in steps so that a series of measurement points are approached one after the other. At each measurement point, the fluid quantity withdrawn is determined on the basis of the data recorded by the flow sensor. Continuous withdrawal with simultaneous recording of the pressure pₑₛₒ in the esophageal catheter and the converted fluid quantity withdrawn, at least for a plurality of measurement points, is also conceivable.

To determine the filling quantity upper limit Nₘₐₓ and the filling quantity lower limit Nₘᵢₙ, the pressure pₑₛₒ and the fluid quantity N in the esophageal catheter can be reduced more slowly, i.e. with a smaller time gradient, than in the previously described method for determining the flow resistance R_{flow}, in which the pressure pₑₛₒ and the fluid quantity N in the esophageal catheter are reduced in pulse-like manner, i.e. with a large time gradient.

In a method for determining the filling quantity upper limit Nₘₐₓ and the filling quantity lower limit Nₘᵢₙ, the fluid quantity N in the esophageal catheter is reduced, for example, at a rate of 8*10-4 mol/min or a rate of 20 ml/min.

While the fluid quantity N and the pressure pₑₛₒ in the esophageal catheter are reduced, both the currently withdrawn fluid quantity ΔNᵢ and the pressure p₁ measured in the esophageal catheter are measured and stored as a pair of measurement values (Δnᵢ, pᵢ). The fluid quantity ΔNᵢ withdrawn from the esophageal catheter is determined either with respect to a fixed start value, for example the upper filling quantity N_{full}, or with respect to a previous measurement point Nⱼ (j<i, in particular j=i−1).

If the pairs of measurement values (Δnᵢ, pᵢ) are plotted as a curve representing the relationship between the pressure pᵢ measured in the esophageal catheter and the fluid quantity Nᵢ in the esophageal catheter associated with the respective pressure pᵢ, this curve usually has a central linear range (“plateau range”) in which the change in pressure pₑₛₒ is in a substantially linear relationship to the change in fluid quantity N.

In particular, the pressure pₑₛₒ changes very little with a change in the fluid quantity N in the linear plateau range, so that the gradient or slope in the linear plateau range of the curve is small. Ideally, the pressure pₑₛₒ in this range is almost constant even with a change in fluid quantity N, so that the gradient is negligible.

Non-linear ranges adjoin the two outer ends of the linear plateau range. In the nonlinear ranges, the pressure pₑₛₒ in the esophageal catheter changes greatly with even small changes in the fluid quantity N in the esophageal catheter. In other words, in the non-linear ranges, the curve describing the pressure pₑₛₒ in the esophageal catheter with respect to the fluid quantity N in the esophageal catheter has positive or negative slopes dp/dN that have a large absolute value.

In the non-linear ranges where the pressure changes significantly with changes in volume, the quantity of measuring fluid in the esophageal catheter N can also be expressed by the volume V of the esophageal catheter when the pressure p is known: N=f(pₑₛₒ, V).

Since the esophageal catheter should preferably be operated in the linear plateau range when used in a patient (“in vivo”) in order to reliably obtain accurate measurement values, a filling quantity upper limit Nₘₐₓ and a filling quantity lower limit Nₘᵢₙ are determined in the following, at which the linear range transitions into one of the non-linear ranges. On the basis of the pressures pₘᵢₙ and pₘₐₓ recorded at the filling quantity upper limit Nₘₐₓ and the filling quantity lower limit Nₘᵢₙ, respectively, the lower limit (minimum filling quantity Nₘᵢₙ) and the upper limit (maximum filling quantity Nₘₐₓ) can also be expressed as volume lower limit (minimum volume) Vₘᵢₙ and as volume upper limit (maximum volume Vₘₐₓ) using the general gas equation.

In one embodiment, the filling quantity lower limit Nₘᵢₙ and the filling quantity upper limit Nₘₐₓ can be determined by numerical evaluation of the previously described curve, which represents the relationship between the pressure p and the fluid quantity N in the esophageal catheter. The numerical evaluation may comprise, for example, determining the slope of the change in pressure pₑₛₒ in the esophageal catheter with respect to a changing fluid quantity N in the esophageal catheter.

In other words, the upper limit Nₘₐₓ of the plateau range is determined such that a linear increase of the detected balloon pressure in the plateau range with increasing quantity of measuring fluid in the balloon probe at the upper limit Nₘₐₓ of the plateau range transitions into a more strongly increasing increase, and that at the upper limit Nₘₐₓ of the plateau range a more strongly decreasing decrease of the detected balloon pressure with decreasing quantity of measuring fluid transitions into a linear decrease of the detected balloon pressure with decreasing quantity of measuring fluid in the plateau range.

Analogously, the lower limit Nₘᵢₙ of the plateau range is determined such that the linear decrease of the detected balloon pressure in the plateau range with decreasing measuring fluid quantity, at the lower limit Nₘᵢₙ of the plateau range transitions with ever decreasing measuring fluid quantity into a more strongly decreasing decrease, and that a more strongly increasing increase of the detected balloon pressure at the lower limit Nₘᵢₙ of the plateau range transitions with increasing measuring fluid quantity into the linear increase of the detected balloon pressure in the plateau range.

In one embodiment, the method comprises, starting from the linear plateau range in which the amount of the gradient dp/dN of the pressure p as a function of the fluid quantity N does not exceed a predefined value Sₘₐₓ (dp/dN≤Sₘₐₓ), identifying those values Nₘᵢₙ and Nₘₐₓ of the fluid quantity N in the esophageal catheter at which the absolute amount of the gradient dp/dN of the pressure pₑₛₒ as a function of the fluid quantity N exceeds the predefined value Sₘₐₓ for the first time (dp/dN>Sₘₐₓ).

The filling quantity lower limit Nₘᵢₙ and filling quantity upper limit Nₘₐₓ determined in this way, which limit the linear plateau range upwards and downwards, can be stored as characteristic properties or characteristic parameters of the esophageal catheter.

If the filling quantity lower limit Nₘᵢₙ and filling quantity upper limit Nₘₐₓ, which limit the linear plateau range of the esophageal catheter, are known, it can be ensured that the esophageal catheter is operated with a filling quantity Nₘᵢₙ<N<Nₘₐₓ which is within the linear range during operation, i.e. during in vivo use in the esophagus of a patient, so that the esophageal catheter reliably provides measurement results with the accuracy required for efficient ventilation.

In one embodiment, the method comprises determining, in addition to the filling quantity lower limit Nₘᵢₙ and filling quantity upper limit Nₘₐₓ, a nominal filling quantity N_{default} with which the esophageal catheter should preferably be filled during operation in order to be able to reliably provide measurement results with the desired accuracy.

When the esophageal catheter is filled with the nominal filling quantity N_{default}, the balloon of the balloon probe of the esophageal catheter should be partially unfolded. However, the balloon of the balloon probe should still be able to be filled further in this state if additional measuring fluid is filled into the balloon probe. N_{default} should therefore lie in a middle portion of the plateau range between Nₘᵢₙ and Nₘₐₓ, so that the balloon probe fills with an increasing quantity of measuring fluid in the balloon probe and continues to deflate with a decreasing quantity of measuring fluid in the balloon probe, so that there is an essentially linear relationship between the pressure p and the quantity N of measuring fluid in the balloon probe. This linear relationship is largely based on the stretchability of the environment, since, as described above, the relationship dp/dV in the linear range between Vₘᵢₙ and Vₘₐₓ is weak.

In one embodiment, the nominal filling quantity N_{default} is fixed such that the nominal filling quantity is a predetermined fraction of the upper filling quantity V_{full}. The nominal filling quantity N_{default} may be, for example, between 30% or ⅓ and 50%, in particular 45%, of the upper filling quantity N_{full}.

In an alternative embodiment, the nominal filling quantity N_{default} is set to the middle of the plateau range. In this case, the following applies: N_{default}=Nₘᵢₙ+ (Nₘₐₓ−Nₘᵢₙ)/2.

The nominal filling quantity N_{default} can also be stored in the storage device for subsequent use.

In one embodiment, the nominal filling quantity N_{default} can be determined by a ventilation device interacting with a system according to the invention or can be set on the ventilation device.

In any of the methods described above, the quantity N of measuring fluid currently present in the balloon probe of the esophageal catheter can be determined by detecting and/or controlling the mass flow q(t) of the measuring fluid into the balloon probe or out of the balloon probe.

When performing the methods described above, it is important in each case that several measurement points, each comprising a pair (pᵢ, ΔNᵢ) of a pressure p₁ and a quantity ΔNᵢ of measuring fluid in the esophageal catheter (measuring fluid quantity) associated with the respective pressure pᵢ in relation to a start value or in relation to a previous measurement value, are traversed, determined and stored for subsequent evaluation.

In one embodiment, the approach of the individual measurement points can be repeated in steps by introducing or withdrawing fluid from or into the esophageal catheter until a predetermined range has been traversed. The predetermined range is selected in particular such that it comprises at least the linear plateau range described above.

The quantity of measuring fluid in the balloon probe is changed in steps between a start value and an end value. The step size of the individual steps can be chosen almost arbitrarily. It is only important that pairs (pᵢ, ΔNᵢ) of pressure in the esophageal catheter and a quantity ΔNᵢ of measuring fluid in the esophageal catheter (“measuring fluid quantity”) associated with the respective balloon pressure pᵢ are determined.

In particular, the step size can be selected so small that the quantity N of measuring fluid in the esophageal catheter is virtually continuously changed.

In one embodiment, to determine the at least one characteristic property of the balloon probe for approaching the respective measurement points, the quantity of measuring fluid in the balloon probe can be changed monotonically in at least two steps from the start value until the end value is reached.

The term “monotonically” means here that the quantity of measuring fluid is changed in the same direction in each step, i.e. that it either continues to decrease or continues to increase between the start value and the end value.

In one embodiment, in order to determine the at least one property characteristic of the balloon probe for approaching the respective measurement points, the quantity of measuring fluid in the balloon probe can be changed in several partial ranges starting from the start value until the end value is reached, wherein the quantity of measuring fluid in the balloon probe is changed monotonically within each partial range and the direction in which the quantity of measuring fluid is changed in each step can be changed between the partial ranges. Thus, the quantity of measuring fluid can increase in each step within a first partial range and decrease in each step within a second partial range. Alternatively, the quantity of measuring fluid can decrease in each step within the first partial range and increase in each step within the second partial range.

In one embodiment, the quantity of measuring fluid in the balloon probe can be reduced monotonically when determining the variables N_{full}, Nₘₐₓ and/or Nₘᵢₙ, in particular starting from a pressure in the balloon probe of +15 hPa to −5 hPa.

In one embodiment, the quantity of measuring fluid in the balloon probe can be increased monotonically during the determination of V_{sys}. In particular, measuring fluid is first withdrawn until an initial pressure of −30 hPA is set. Then measuring fluid is introduced or let into the balloon probe until the pressure in the balloon probe has increased to −20 hPa or the negative pressure in the balloon probe has decreased accordingly. In a subsequent step, measuring fluid is again introduced or let into the balloon probe until the pressure in the balloon probe has increased to a pressure of −10 hPa.

In one embodiment, the change in the fluid quantity in the esophageal catheter can be determined by detecting and/or controlling the mass flow of measuring fluid into the esophageal catheter or out of the esophageal catheter. In particular, the change in the filling quantity can be determined by integrating the mass flow over the time between a starting point and the respective measurement point, as described above.

In one embodiment, starting from a starting state in which a starting pressure p_{A} has been determined, measuring fluid can be introduced into the balloon probe or withdrawn from the balloon probe until a final pressure p_{E} is reached. The fluid quantity ΔN that has been introduced into or withdrawn from the balloon probe can then be calculated as the integral over the mass flow q(t) recorded between the starting pressure p_{A} and the final pressure p_{E}.

In one embodiment, at least two measurement cycles can be carried out in succession. The quality and reliability of the characterization can be further improved by carrying out several measurement cycles.

In particular, each measurement cycle can comprise at least the steps of filling the esophageal catheter with measuring fluid ex vivo, in particular before placing the esophageal catheter in the esophagus, and detecting a pressure prevailing in the esophageal catheter.

In order to create the same starting conditions before each measurement cycle, complete emptying of measuring fluid from the esophageal catheter can take place between successive measurement cycles.

Alternatively, no or at least no complete emptying of measuring fluid from the esophageal catheter can take place between successive measurement cycles. The procedure can be accelerated by not completely emptying the esophageal catheter. In this regard, it can be helpful to know the system volume V_{sys} of the esophageal catheter.

A respective measurement range can be associated with each measurement cycle. A first measurement range for a preceding measurement cycle may differ from a second measurement range for a subsequent measurement cycle. In particular, a preceding measurement cycle can define the measurement range for a subsequent measurement cycle.

In one embodiment, the interval between successive measurement points can be set differently for a subsequent measurement cycle than for a previous measurement cycle. By varying the intervals between successive measurement points, the quality and reliability of the characterization can be further improved.

For example, an efficient approximation algorithm, such as the Newton algorithm, can be used.

In one embodiment, the method may comprise determining a viscoelasticity of the esophageal catheter, in particular of the balloon probe of the esophageal catheter. In particular, the method may comprise introducing a predetermined quantity of measuring fluid into the esophageal catheter and, after introducing the predetermined quantity of measuring fluid into the esophageal catheter, determining the time required until the pressure in the esophageal catheter, which is predefined by the quantity of measuring fluid in the esophageal catheter, has adjusted to a stable value. The predetermined quantity of measuring fluid introduced into the esophageal catheter may be predefined in particular by a predetermined positive pressure in the esophageal catheter.

In one embodiment, a method according to the invention for the automated characterization of an esophageal catheter may comprise all or some of the (partial) methods described above, wherein each of the (partial) methods is intended to determine at least one characteristic property or parameter of the esophageal catheter.

The (partial) methods or processes described above can be combined with each other as desired and carried out in any order.

In one embodiment, the previously determined characteristic properties or parameters of the esophageal catheter can be transmitted to a ventilation device in which the esophageal catheter is used.

The characteristic properties or parameters of the esophageal catheter can be transmitted to the ventilation device, for example, via a cable or radio connection, e.g. WLAN or Bluetooth.

The ventilation device can then take the previously determined characteristic properties or parameters of the esophageal catheter into account in evaluating the measurement values provided by the balloon probe of the esophageal catheter in order to optimize the control of automated ventilation. Defects, in particular leaks, of the esophageal catheter can also be detected quickly and reliably by way of the previously determined characteristic properties or parameters of the esophageal catheter.

In one embodiment, the characteristic properties or parameters of the esophageal catheter can be output via an output device, for example a display device, e.g. a screen and/or a printer. A user can thus document the characteristic properties or parameters of the esophageal catheter and check them for plausibility. An esophageal catheter identified as defective can be discarded before it is used in ventilating a patient.

In one embodiment, the method may comprise providing the esophageal catheter with at least one identifier that is associated with the at least one stored characteristic property or parameter, so that a fixed connection or association is created between the esophageal catheter and the at least one stored characteristic property or parameter.

In this way, the characteristic parameter is inseparably associated with the esophageal catheter and can be uniquely determined by way of the at least one identifier applied to or on the esophageal catheter. In this manner, incorrect associations between esophageal catheters and characteristic properties or parameters can be reliably prevented.

In one embodiment, the identifier may comprise a code on the basis of which the stored characteristic parameter can be ascertained. For example, each esophageal catheter may be provided with an individual labeling and the characteristic parameter may be stored together with the individual labeling. An identifier applied to the esophageal catheter corresponds to the individual labeling, so that the characteristic parameter can be retrieved using the identifier applied to the esophageal catheter.

In one embodiment, the esophageal catheters can be divided into classes, so that each characteristic parameter, or each individual labeling, as the case may be, belongs to one of the classes. The identifier on the catheter may be associated with a class to which a respective esophageal catheter belongs, or the respective class to which a respective esophageal catheter belongs may be determined on the basis of the identifier.

The classes may comprise, for example, at least the two classes “suitable” and “not suitable” in order to be able to distinguish suitable esophageal catheters from unsuitable, for example defective, esophageal catheters.

The classes may also comprise more than two classes, for example different quality classes, or classes corresponding to different purposes and/or measurement ranges of the catheters.

In one embodiment, the output device can be adapted to display the characteristic properties or parameters as a machine-readable code, for example as a bar code or as a QR code.

In one embodiment, the output device can be adapted to output the characteristic properties or parameters in a form that permits the characteristic properties or parameters to be applied to the esophageal catheter or its packaging. For example, the characteristic properties or parameters can be output in the form of a sticker that can be attached to the esophageal catheter or its packaging.

In one embodiment, the output device can be designed to apply a machine-readable code directly to the esophageal catheter, for example with the aid of a laser beam. The machine-readable code can also be transmitted to an RFID carrier (RFID=Radio Frequency Identification) or an NFC carrier (NFC=Near Field Communication), which is or will be attached to the esophageal catheter or to a packaging of the esophageal catheter.

In one embodiment, a system according to the invention may comprise a device for providing the esophageal catheter with at least one identifier that is associated with the at least one stored characteristic property.

In one embodiment, an esophageal catheter according to the invention may comprise at least one identifier that is associated with the at least one stored characteristic property.

In this way, the previously determined characteristic properties or parameters of the esophageal catheter are fixedly connected to the esophageal catheter and can be read out quickly and reliably by a ventilation device with which the esophageal catheter is used. Transmission errors, which can occur during manual transmission of the characteristic properties or parameters, can be reliably avoided in this way.

In the following, embodiments of the invention will be described in more detail with reference to the accompanying drawing figures.

### BRIEF DESCRIPTION OF THE DRAWINGS

FIG. 1 shows a schematic representation of an esophageal catheter with a catheter tube that can be inserted into the esophagus of a patient and with a system according to the invention for automated characterization of the esophageal catheter.

FIG. 2 shows a flowchart illustrating the course of a method for determining a system volume V_{sys}.

FIG. 3 shows a flowchart illustrating the course of a method for determining the flow resistance R of the esophageal catheter.

FIG. 4 shows a flowchart illustrating the course of a method for detecting leakage in the esophageal catheter.

FIG. 5 shows a flowchart illustrating the course of a method for determining the upper limit Nₘₐₓ and the lower limit Nₘᵢₙ of a linear range of the esophageal catheter.

FIG. 6 shows a diagram that exemplifies the relationship between a pressure pₑₛₒ measured in the esophageal catheter 48, which is plotted on the ordinate, and the fluid quantity N in the esophageal catheter 2, which is plotted on the abscissa and is associated with the respective measured pressure pₑₛₒ.

FIG. 7 shows a flowchart illustrating a method for the automated characterization of an esophageal catheter that can be inserted into the esophagus of a patient to be ventilated, which comprises the (partial) methods described in FIGS. 2 to 5.

FIG. 8 shows a schematic representation of the essential elements of a ventilation device according to the invention with a system according to the invention for the automated characterization of an esophageal catheter.

### DETAILED DESCRIPTION

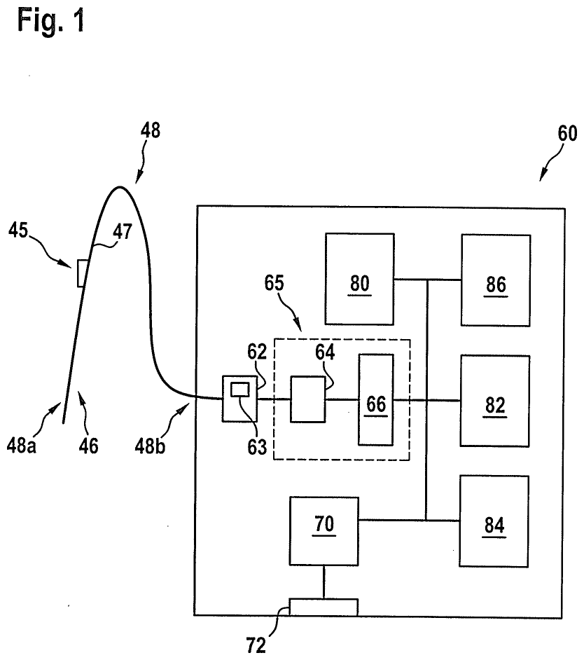

FIG. 1 shows a simplified schematic representation of an esophageal catheter 48 and a system 60 for automated characterization (“characterization system 60”) of the esophageal catheter 48.

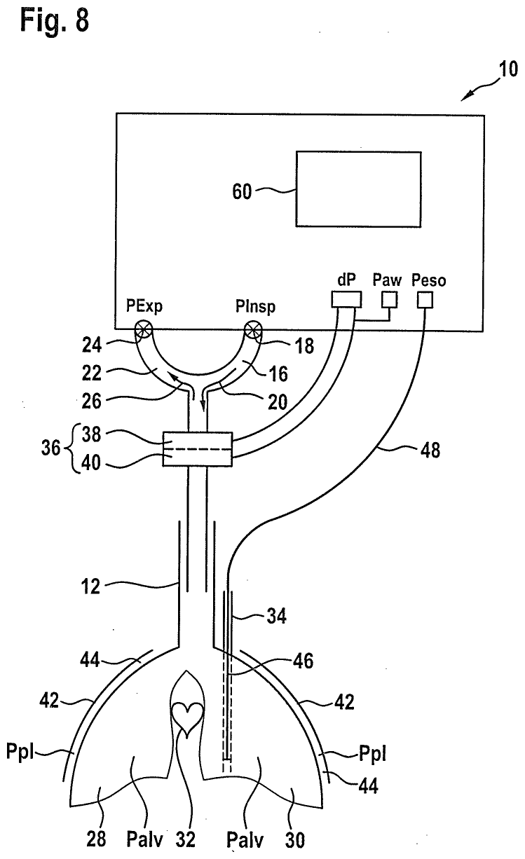

The esophageal catheter 48 comprises a catheter tube 47 insertable into an esophagus 34 (see FIG. 8) of a patient.

A balloon probe 46 is attached to a first, proximal end 48a of the catheter tube 47.

A second, distal end 48b of the catheter tube 47 is connected to the characterization system 60 of the esophageal catheter 48 in such a way that the esophageal catheter 48 can be acted upon by, in particular filled with, a measuring fluid, in particular air, by the characterization system 60. The characterization system 60 is also capable of withdrawing the measuring fluid from the esophageal catheter 48 in order to empty the esophageal catheter 48.

The connection between the second end of the catheter tube 47 and the characterization system 60 is releasable, so that the esophageal catheter 48 is selectively connectable to and separable from the characterization system 60.

The characterization system 60 comprises a pumping device 65 that is configured to selectively introduce measuring fluid into the esophageal catheter 48 and withdraw measuring fluid from the esophageal catheter 48. The pumping device 65 is in fluid communication with the catheter tube 47 of the esophageal catheter 48 and comprises, in particular, a fluid pump 66 and a valve 64.

The characterization system 60 further comprises a flow sensor 62, in particular a mass flow sensor 62, which is configured to determine the quantity of measuring fluid introduced into the balloon probe 46 and/or the quantity of measuring fluid withdrawn from the balloon probe 46.

The respective quantity of measuring fluid introduced into the balloon probe 46 or withdrawn from the balloon probe 46 can be determined in particular by measuring a flow q of measuring fluid through the flow sensor 62 and integrating the flow q measured by the flow sensor 62 over a period of time between a start time and an end time. The flow sensor 62 can, for example, be designed such that it includes at least one pressure sensor 63 in order to measure a differential pressure Δp and to determine the flow of the measuring fluid from the measured differential pressure Δp. Depending on the design of the pressure sensor or by providing an additional pressure sensor, a sensor based on the differential pressure principle can also determine the corresponding mass flow in addition to a volume flow. The flow sensor 62 can in particular be designed such that it can also be used to determine (in addition to a pressure difference) the esophageal balloon pressure pₑₛₒ prevailing in the balloon probe 46, so that a volume flow V(t) detected by the flow sensor at a particular point in time can be converted into a corresponding mass flow q(t). There may also be provided an additional pressure sensor 63 in order to be able to determine the esophageal balloon pressure pₑₛₒ prevailing in the balloon probe 46 and thus determine the corresponding mass flow q(t).

The flow sensor 62 can also operate according to a different principle, for example according to a thermal principle, in particular according to a thermal principle in which the mass flow of the fluid flowing through the sensor is determined by way of the heat dissipated by transport of fluid when a certain thermal power is absorbed. If such a sensor primarily detects a volume flow, an additional pressure sensor 63 would have to be provided in order to be able to determine the esophageal balloon pressure pₑₛₒ prevailing in the balloon probe 46 and to thus determine the corresponding mass flow q(t).

The flow sensor 62 can be, for example, a MEMS sensor (MEMS=micro-electromechanical system) which measures the mass flow based on a thermal principle. MEMS sensors are particularly space-saving and inexpensive.

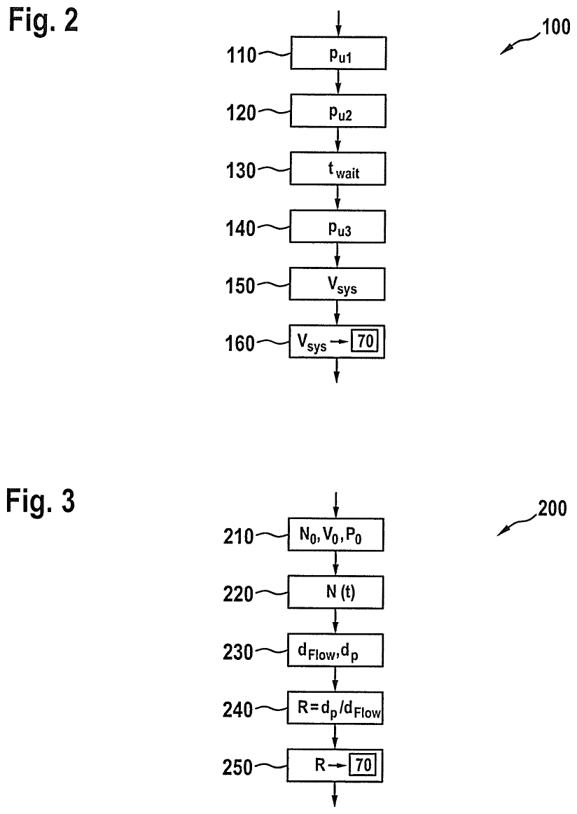

The characterization system 60 also comprises four controllers 80, 82, 84, 86, each adapted to perform a method for determining a characteristic property or parameter of the esophageal catheter 48 connected to the characterization system 60. The details of the various methods will be described below with reference to FIGS. 2 to 5.

The controllers 80, 82, 84, 86 may be implemented as independent components “in hardware”. Two or more controllers 80, 82, 84, 86, in particular all controllers 80, 82, 84, 86, can also be integrated in a common component or in a common group of components. The controllers 80, 82, 84, 86 may be integrated in a ventilation device.

The controllers 80, 82, 84, 86 may also be implemented as a computer program product, i.e. by a corresponding software program which is executed on a processor, in particular a microprocessor or microcontroller. In this case, the software can be provided on a suitable local storage medium or a storage medium that can be accessed via a network. The software contains instructions coded as a computer program which, when the software is loaded into a RAM memory of the processor and translated into machine language, causes the processor to execute the procedures described in more detail herein. Mixed forms between an implementation in hardware and an implementation in software are possible as well.

The characterization system 60 also comprises a storage device 70 that is configured to store at least one characteristic property of the esophageal catheter 48 that has been determined by the characterization system 60.

FIG. 2 shows a flowchart illustrating the course of a method 100 for determining a system volume V_{sys} of an esophageal catheter 48. The system volume V_{sys} of an esophageal catheter 48 is the volume thereof when the balloon probe 46 is completely collapsed. The system volume V_{sys} is determined substantially by the volume of the catheter tube 47. The system volume V_{sys} may also include the volumes of other elements of the esophageal catheter 48 that are in fluid communication with the catheter tube 47, such as couplings, connectors, etc., which are not shown in FIG. 1.

In a first step 110, the esophageal catheter 48 is evacuated by means of the pumping device 65. In doing so, a first negative pressure pᵤ₁ of, for example, pᵤ₁=−30 hPa is set in the esophageal catheter 48 by the pumping device 65. The pressure specifications given here and in the following always refer to the ambient pressure p_{amb}, i.e. a negative pressure pᵤ₁ of −30 hPa means that the pressure pₑₛₒ in the esophageal catheter 48 is 30 hPa below the ambient pressure p_{amb}:pᵤ₁=p_{amb}−30 hPa.

In a second step 120, the negative pressure in the esophageal catheter 48 is reduced by introducing fluid into the esophageal catheter 48, i.e., the pressure in the esophageal catheter 48 is increased. For example, a second negative pressure pᵤ₂ of −20 hPa is set. For this purpose, the fluid can be allowed to flow into the esophageal catheter 48 by opening the valve 64. The fluid can also be actively pumped into the esophageal catheter 48 by the fluid pump 66.

After a predetermined waiting time t_{wait} has elapsed, which may be between 2 s and 10 s, in particular about 3 s (third step 130), the negative pressure in the esophageal catheter 48 is further reduced in a fourth step 140 by reintroducing fluid into the esophageal catheter 48. In particular, a third negative pressure pᵤ₃ of, for example, −10 hPa is set.

While the negative pressure in the esophageal catheter 48 is reduced from the second negative pressure pᵤ₂ to the third negative pressure pᵤ₃, the pressure curve p(t) in the esophageal catheter 48 and the mass flow q(t) and/or volume flow V(t) of the measuring fluid, for example air, flowing into the esophageal catheter 48 are measured. Insofar as the flow sensor 62 used for this purpose also detects the pressure pₑₛₒ currently prevailing in the esophageal catheter 48, or the pressure pₑₛₒ in the esophageal catheter 48 (or the density of the fluid in the esophageal catheter 48) is known from another measurement, the corresponding mass flow q(t) can be determined from a volume flow V(t) detected by the flow sensor, and can be used to determine the system volume V_{sys}.

The pressure dependency of the relationship between mass flow q(t) and volume flow v(t) can be negligible in the context of the pressure changes applied here relative to the required accuracy. In this case, a volume flow v(t) measured by the flow sensor 62 can also be used directly to determine the system volume V_{sys} and other characteristic properties of the esophageal catheter 48 without determining the associated mass flow q(t).

The fluid quantity ΔN flowing into the esophageal catheter 48 results from an integration of the mass flow q(t) measured by the flow sensor 62 over time between a starting point pₑₛₒ=pᵤ₂ and an end point pₑₛₒ=pᵤ₃:

ΔN=∫q(t)dt.

From these measurement variables, it is possible, using the general gas equation

PV=N kT,
to then determine the system volume V_{sys} in step 150 as a characteristic property of the esophageal catheter 48:

$${V}_{sys} = Δ N kT / ( {p}_{u 3} - {p}_{u 2} ) ,$$

wherein
p is the pressure,
V is the volume,
V_{sys} is the system volume,
N is the quantity of substance or fluid,
ΔN is the fluid quantity introduced into the esophageal catheter,
k is the Boltzmann constant, and
T is the current temperature.

The system volume V_{sys} determined in this way is then stored in the storage device 70 in a subsequent step 160 as a characteristic property or characteristic parameter of the esophageal catheter 48.

FIG. 3 shows a flowchart illustrating the course of a method 200 for determining the flow resistance R of the esophageal catheter 48.

In a first step 210, a predetermined filling level of the esophageal catheter 48 is set. The predetermined filling level may be defined by a predefined fluid quantity N₀, a predefined filling volume V₀, or by a predefined pressure p₀ in the esophageal catheter 48.

After a predetermined filling level has been set in the esophageal catheter 48, the fluid quantity N(t) in the esophageal catheter 48 is changed in pulse-like manner, i.e. with a relatively large time gradient, by the pumping device 65 in a subsequent step 220. Thereby, within a relatively short period of time Δt, additional measuring fluid is introduced into the esophageal catheter 48 or withdrawn from the esophageal catheter 48, so that the pressure pₑₛₒ in the esophageal catheter 48 changes rapidly, i.e. with a large time gradient.

For example, the fluid quantity N(t) in the esophageal catheter 48 can be changed with a gradient of 3.6*10−3 mol/min (or 90 ml/min).

The period of time Δt in which the fluid quantity N(t) in the esophageal catheter 48 is changed may have a duration in the range of 0.2 s to 1 s.

In step 230, during the period of time Δt, i.e., while the fluid quantity N in the esophageal catheter 48 is changed with a large time gradient, the temporal change of the mass flow dq and the pressure change dpt caused by the change of the fluid quantity in the esophageal catheter 48 are measured using the flow sensor 62.

In step 240, the flow resistance R_{flow} of the esophageal catheter 48 is then determined as the quotient of the pressure change dp measured in step 230 and the temporal change of the mass flow dq measured in step 230:

R_{flow}=dp/dq.

The flow resistance R_{flow} determined in this manner is then stored in the storage device 70 in the subsequent step 250 as a characteristic property or characteristic parameter of the esophageal catheter 48.

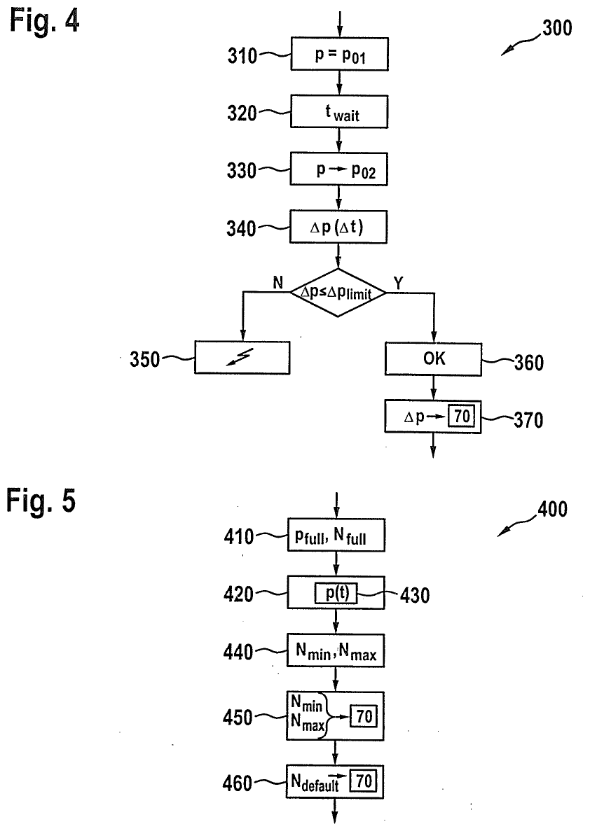

FIG. 4 shows a flowchart illustrating the course of a method 300 for detecting a leakage in the esophageal catheter 48.

In a first step 310, the esophageal catheter 48 and in particular the balloon probe 46 are brought (“inflated”) to a first positive pressure pₒ₁, for example a first positive pressure pₒ₁ of 28 hPa, by means of the pumping device 65. Here too, the pressures refer to the ambient pressure p_{amb}. A positive pressure pₒ₁ of 28 hPa thus means that the pressure in the esophageal catheter 48 is 28 hPa greater than the ambient pressure p_{amb}:pₒ₁=p_{amb}+28 hPa.

In a second step 320, a predetermined waiting time t_{wait} is waited to allow complete pressure equalization within the esophageal catheter 48. The predetermined waiting time t_{wait} may be between 2 s and 10 s; in particular, the predetermined waiting time t_{wait} may be about 3 s.

After the predetermined waiting time t_{wait} has elapsed, the pressure pₑₛₒ in the esophageal catheter 48 is reduced in a third step 330 to a first positive pressure pₒ₂, which is less than the first positive pressure pₒ₁ (pₒ₂<pₒ₁).

In a subsequent, fourth step 340, the pressure pₑₛₒ in the esophageal catheter 48 is measured over a predetermined period of time Δt without any additional measuring fluid being introduced into or withdrawn from the esophageal catheter 48 during this period of time Δt. The predetermined period of time Δt may comprise periods of time Δt in the range from 5 s to 20 s, in particular a period of about 10 s.

If the pressure drop Δp=pₒ₂−pₒ₃ in the esophageal catheter 48 measured within the predetermined period of time Δt exceeds a predetermined limit value Δpₗᵢₘᵢₜ (Δp>Δpₗᵢₘᵢₜ), a message is output to the user in step 350, that the system of esophageal catheter 48 and characterization system 60 is leaking, which may be due to a defect in the esophageal catheter 48 or a leaky connection between the esophageal catheter 48 and the characterization system 60.

If the pressure drop Δp=pₒ₂−pₒ₃ in the esophageal catheter 48 measured within the predefined period of time Δt does not exceed the specified limit value Δpₗᵢₘᵢₜ (Δp≤ Δpₗᵢₘᵢₜ), the esophageal catheter 48 is not defective and can be used to control the ventilation of a patient (“in vivo”). A corresponding message is issued to the user in step 360.

Setting the first positive pressure pₒ₁ in preparation for the actual leakage measurement is optional. In other words, in an alternative method, the setting of a first pressure pₒ₁ before the actual measurement can be dispensed with. In this case, the predetermined period of time Δt over which the pressure drop in the esophageal catheter 48 can be observed is generally longer than in the method described hereinbefore, in which a first positive pressure pₒ₁>pₒ₂ is set in preparation for the actual leakage measurement.

A method for detecting a leakage in the esophageal catheter 48 can also be performed with negative pressure, instead of positive pressure as described hereinbefore. In this case, instead of the aforementioned positive pressures pₒ₁, pₒ₂, pₒ₃ in the esophageal catheter 48, various negative pressures pᵤ₁, pᵤ₂, pᵤ₃, i.e. pressures that are less than the ambient pressure p_{amb}, are set in succession. The negative pressure in the esophageal catheter 48 is thereby reduced in each step, i.e. the pressure in the esophageal catheter 48 is increased and thereby brought closer to the ambient pressure p_{amb}.

A method with positive pressures pₒ₁, pₒ₂, pₒ₃ and a method with negative pressures pᵤ₁, pᵤ₂, pᵤ₃ may also be performed sequentially, and the two methods may be performed in any order. The volumes introduced into the esophageal catheter 48 while performing the methods and the volumes withdrawn from the esophageal catheter 48 can be measured and compared with each other. A difference in these volumes may be used to infer leakage of the system of esophageal catheter 48 and characterization system 60.

Optionally, in an additional step 370, the measured pressure drop Δp or the pressure drop rate Δp/Δt can be stored in the storage device 70 as a characteristic property or characteristic parameter of the esophageal catheter 48.

If the pressure drop rate Δp/Δt measured for an esophageal catheter 48 is known, this can be taken into account during subsequent use of the esophageal catheter 48 on the patient (“in vivo”) and, if necessary, compensated for by calculation or by repeated, in particular periodic, refilling of the esophageal catheter 48. The esophageal catheter 48 can also be recognized as defective during operation if the pressure drop rate Δp/Δt determined during use on the patient exceeds the pressure drop rate Δp/Δt previously determined as a characteristic parameter of the esophageal catheter 48 by more than a predetermined tolerance.

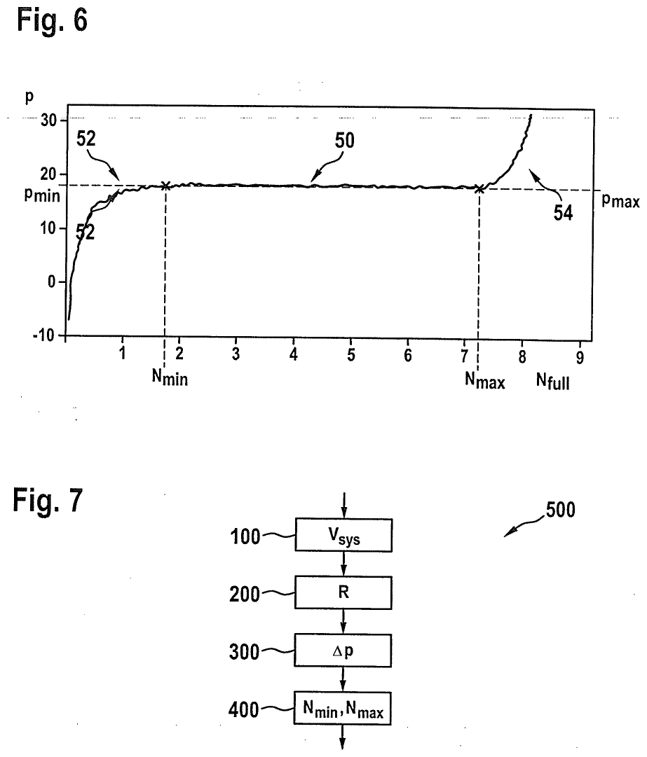

The balloon probe 46 of an esophageal catheter 48 has a linear range 50 (see FIG. 6), in which the pressure pₑₛₒ within the esophageal catheter 48 does not change significantly due to a corresponding expansion of the balloon probe 46 when the filling quantity N is changed within this range which is limited by the filling quantities Nₘᵢₙ and Nₘₐₓ (Nₘᵢₙ<N<Nₘₐₓ).

FIG. 5 shows a flowchart illustrating the course of a method 400 for determining the upper limit Nₘₐₓ and the lower limit Nₘᵢₙ of the linear range 50.

To perform this method, the esophageal catheter 48 is first filled with measuring fluid in step 410, for example using the pumping device 65, until a predetermined upper pressure p_{full} is reached within the esophageal catheter 48. The predetermined upper pressure p_{full} corresponds to an upper filling quantity N_{full} or an upper filling volume V_{full} of the esophageal catheter 48, which results from the upper filling quantity N^{full} according to the previously mentioned general gas equation pV=N kT.

The predefined upper pressure p_{full} is above the expected linear range 50. In this state, the balloon probe 46 of the esophageal catheter 48 is “inflated” and filled with measuring fluid without the risk of the balloon probe 46 being damaged by excessive pressure. The corresponding point (p_{full}, N_{full}) on the balloon pressure/measuring fluid quantity curve shown in FIG. 6 lies to the right of the linear range 50 of the curve, as a range 54 of the curve in which the curve rises non-linearly and steeply as the quantity N of measuring fluid in the esophageal catheter 48 increases.

The predetermined upper pressure p_{full}, for example, may be in a range from 10 hPa to 20 hPa, in particular the predetermined upper pressure p_{full} can be a pressure of about 15 hPa.

After the pressure pₑₛₒ in the esophageal catheter 48 has been brought to the predetermined upper pressure p_{full}, the pressure pₑₛₒ in the esophageal catheter 48 is gradually reduced in the subsequent step 420 by withdrawing fluid, in particular with the aid of the pumping device 65. In this process, the fluid can be drained from the esophageal catheter 48 by opening the valve 64. The fluid can also be actively pumped out of the esophageal catheter 48 by the fluid pump 66.

In the method illustrated in FIG. 5, the fluid quantity N in the esophageal catheter 48 is reduced in particular until a negative pressure, for example a negative pressure of −5 hPa, is measured in the esophageal catheter 48 with respect to the ambient pressure p_{amb}.

In the method for determining the linear range 50, the pressure pₑₛₒ and the fluid quantity N in the esophageal catheter 48 are reduced more slowly, i.e. with a lower time gradient, than in the previously described method 200, in which the flow resistance R is determined.

For example, the fluid quantity N in the esophageal catheter 48 is reduced at a rate of 8*10−4 mol/s or a rate of 20 ml/s.

While the fluid quantity N and the pressure pₑₛₒ in the esophageal catheter 48 are reduced, both the currently withdrawn fluid quantity N(t) and the respective pressure p(t) measured in the esophageal catheter 48 are measured and stored in step 430.

FIG. 6 shows a diagram which, by way of example, illustrates on the ordinate (y axis) the pressure pₑₛₒ measured in the esophageal catheter 48 and on the abscissa (x axis) the fluid quantity N in the esophageal catheter 2 respectively associated with the measured pressure pₑₛₒ. The fluid quantity N in the esophageal catheter 48 is obtained by integrating the fluid or mass flow q(t) measured by the flow sensor 62 over the time between a start time t₁ and an end time t₂:

ΔN=∫q(t)dt.

When performing the method described above, in which fluid is continuously withdrawn from the esophageal catheter 2, the curve shown in FIG. 6 is traversed from a high filling quantity N_{full} to a low filling quantity, i.e. from right to left.

As shown in FIG. 6, the curve illustrating the pressure pₑₛₒ measured in the esophageal catheter 48 in conjunction with the fluid quantity N in the esophageal catheter 2 associated with the respective pressure pₑₛₒ has a central linear range (“plateau range”) 50 in which the pressure pₑₛₒ changes very little with a change in the fluid quantity N, and the change in the pressure p is essentially in a linear relationship to the change in the fluid quantity N.

The slope or gradient of the curve describing the relationship between the pressure pₑₛₒ and the fluid quantity N is small; ideally, the pressure pₑₛₒ is almost constant even when the fluid quantity N changes.

Adjacent to the two ends of the linear range 50 are non-linear ranges 52, 54 in which the pressure pₑₛₒ in the esophageal catheter 48 strongly changes even with small changes in the fluid quantity N in the esophageal catheter 2, i.e. ranges in which the pressure pₑₛₒ in the esophageal catheter 2 changes greatly when the fluid quantity N in the esophageal catheter 2 is changed. In the non-linear ranges 52, 54, the curve describing the pressure pₑₛₒ in the esophageal catheter 2 with reference to the fluid quantity N in the esophageal catheter 2 has large slopes dp/dN.

When used in a patient (“in vivo”), the esophageal catheter 48 should be operated in the linear range 50.

In a subsequent step 440, the minimum filling quantity Nₘᵢₙ and the maximum filling quantity Nₘₐₓ are determined at which the linear range 50 transitions into one of the non-linear ranges 52, 54. Here too, the minimum filling quantity Nₘᵢₙ and the maximum filling quantity Nₘₐₓ can be expressed as the minimum volume Vₘᵢₙ and the maximum volume Vₘₐₓ on the basis of the respective pressures pₘᵢₙ and pₘₐₓ detected.

The minimum filling quantity Nₘᵢₙ and the maximum filling quantity Nₘₐₓ can be determined in particular by numerical evaluation of the curve shown in FIG. 6, illustrating the pressure pₑₛₒ versus the fluid quantity N in the esophageal catheter 2. This may, for example, comprise determining the slope of the pressure p in the esophageal catheter 2 with respect to the changing fluid quantity N. In particular, the method may comprise, starting from the linear range 50 in which the magnitude of the slope dp/dN of the pressure p as a function of the fluid quantity N does not exceed a predetermined value Sₘₐₓ (dp/dN≤ Sₘₐₓ), identifying those values Nₘᵢₙ and Nₘₐₓ of the fluid quantity N in the esophageal catheter 2 at which the magnitude of the slope dp/dN of the pressure p as a function of the fluid quantity N exceeds the predetermined value Sₘₐₓ for the first time (dp/dN>Sₘₐₓ).

The filling quantities Nₘᵢₙ and Nₘₐₓ determined in this way, which limit the linear range 50 upwards and downwards, are then stored in the storage device 70 in a subsequent step 450 as characteristic properties or characteristic parameters of the esophageal catheter 48.

If the filling quantities Nₘᵢₙ and Nₘₐₓ limiting the linear range 50 of the esophageal catheter 2 are known, the esophageal catheter 2 can be operated “in use”, i.e. when used in a patient (“in vivo”) with a filling quantity N that is within the linear range 50.

In any of the methods described, the current quantity N of measuring fluid in the balloon probe 46 of the esophageal catheter 48 can be determined by detecting and/or controlling the mass flow q(t) of measuring fluid into or out of the balloon probe 46.

This can be done in particular such that, starting from a starting state in which a starting pressure p_{A} is detected, measuring fluid is introduced into or withdrawn from the balloon probe 46 until a final pressure p_{E} is detected, and the integral is calculated over the mass flow q(t) detected between the starting pressure p_{A} and the final pressure p_{E}, as described before.

This procedure can be repeated in steps until a range has been traversed that contains at least the linear range. The step size of the individual steps can be selected as desired. In particular, the step size can be selected so small that the quantity N of measuring fluid in the balloon probe 46 of the esophageal catheter 48 is determined virtually continuously.

When carrying out the methods, it is important that several pairs of pressure pₑₛₒ and the respectively associated quantity N of measuring fluid in the esophageal catheter 48 (measuring fluid quantity) are traversed, determined and stored for subsequent evaluation.

A method according to the invention for the automated characterization of an esophageal catheter 48 may comprise all or some of the (partial) methods described above.

In particular, a method according to the invention for the automated characterization of an esophageal catheter 48 may comprise performing the previously described (partial) methods in succession.

FIG. 7 shows a flowchart illustrating such a method 500 according to the invention. The method comprises the previously described method 100 for determining the system volume V_{sys} of the esophageal catheter 48, the method 200 for determining the flow resistance R of the esophageal catheter 48, the method 300 for detecting a leakage in the esophageal catheter 48, and the method 400 for determining the upper limit Nₘₐₓ and the lower limit Nₘᵢₙ of a linear range of the esophageal catheter 48.

The sequence in which the individual (partial) methods 100, 200, 300, 400 are carried out is not necessarily predetermined. In particular, it can be changed as required. For example, it may be advantageous to perform the method 300 for detecting a leakage in the esophageal catheter 48 as the first process in order to be able to quickly detect and sort out a defective esophageal catheter 48.

Also, a method 500 according to the invention may comprise only a selected subset of the previously described (partial) methods 100, 200, 300, 400. That is, (partial) methods 100, 200, 300, 400 that relate to the determination of characteristic properties or parameters of the esophageal catheter 48 that are not needed for subsequent use of the esophageal catheter 48 may be omitted.

After the desired (partial) methods 100, 200, 300, 400 have been carried out, the characteristic properties or parameters of the esophageal catheter 48 determined in the manner described above are stored in the storage device 70.

The characteristic properties or parameters of the esophageal catheter 48 can be transmitted from there to a ventilation device 10 (see FIG. 8), in which the esophageal catheter 48 is used to control automated ventilation of a patient.

The characteristic properties or parameters of the esophageal catheter 48 can be transmitted to the ventilation device, for example, via a cable or radio connection, for example WLAN or Bluetooth.

The characteristic properties or parameters of the esophageal catheter 48 can also be output via an output device 72, for example a display device (“screen”) and/or a printer.

In particular, the characteristic properties or parameters of the esophageal catheter 48 may be fixedly connected to the esophageal catheter 48 to prevent erroneous association of characteristic properties or parameters to a wrong esophageal catheter 48 and/or loss of the characteristic properties or parameters.

The output device 72 can be designed, for example, to output the characteristic properties or parameters as a machine-readable code 45, in particular as a bar code 45 or as a QR code 45 in a form that enables the code 45 to be attached to the esophageal catheter 48 or its packaging. For this purpose, the machine-readable code 45 can be output, for example, in the form of a sticker that can be attached to the esophageal catheter 48 or its packaging.

The output device 72 may also be configured to apply the machine-readable code 45 directly to the esophageal catheter 48, for example by means of a laser beam. The machine-readable code 45 may also be transmitted on an RFID carrier or an NFC carrier that is or will be attached to the esophageal catheter 48 or to a packaging of the esophageal catheter 48.

In this way, the characteristic properties or parameters are fixedly connected to the esophageal catheter 48 and can be read out quickly and reliably by a ventilation device in which the esophageal catheter 48 is used.

FIG. 8 shows, in a highly schematized representation and in the form of a block diagram, the essential elements of a ventilation device 10 according to the invention with a characterization system 60 according to the invention for automated characterization of an esophageal catheter 48.

The ventilation device 10 is shown in FIG. 8 in a state with intubated trachea 12 of a ventilated patient. In addition to the trachea 12, the lung lobes 28, 30, the heart 32, the esophagus 34 and the thoracic wall 42 of the patient are shown very schematically in FIG. 8. The tube 14 of the ventilation device 10 is inserted, generally through the patient's mouth opening which is not shown, a short distance into the trachea 12 to supply breathing gas to the airway. Exhaled air is also discharged through tube 14, which branches at its upstream end into a first end 16 and a second end 22. The first end 16 is connected via an airway inlet valve 18 to an airway inlet port of the ventilation device 10 for applying an inspiration pressure PInsp. In the open position of the airway inlet valve 18, the inspiration pressure PInsp is applied to the airway. The second end 22 is connected via an airway outlet valve 24 to an airway outlet port of the ventilation device 10 for application of an expiration pressure PExp. In the open position of the airway outlet valve 24, the expiration pressure PExp is applied to the airway.

Both the inspiration pressure PInsp and the expiration pressure PExp are generated by the ventilation device 10 according to predetermined time patterns, such that inspiratory breathing gas flows toward the patient's lungs 28, 30 during an inspiration phase, as indicated by arrow 20 in FIG. 8, and expiratory breathing gas flows back from the patient's lungs 28, 30 during an expiration phase, as indicated by arrow 26. Normally, the airway inlet valve 18 remains open during the inspiration phase, and the inspiration pressure PInsp—which is usually greater than the expiration pressure PExp—is applied to the airway inlet. During the expiration phase, the airway inlet valve 18 is closed and the airway outlet valve 24 is open. Then, the expiration pressure PExp is applied to the airway inlet.

In the context of the present invention, any forms of known ventilation modes can be used, for example, pressure-controlled ventilation modes, volume-controlled ventilation modes, or ventilation modes in which pressure-controlled and volume-controlled aspects are combined. In addition to purely machine-controlled forms of ventilation, in which the time course of the inspiration pressure PInsp and possibly also of the expiration pressure PExp are determined by the ventilation device 10, it is also conceivable to have forms of ventilation in which the patient's spontaneous breathing efforts can either support the machine ventilation or the machine ventilation serves to support the patient's spontaneous breathing efforts. In such forms of ventilation, the time course of inspiration pressure PInsp or expiration pressure PExp and frequently also the position of inlet valve 18 or outlet valve 24 are not determined solely by the ventilation device 10, but are influenced by the patient's spontaneous breathing efforts.

The calibration of an esophageal catheter 48 with a balloon probe 46, which can be introduced into the esophagus 34, for detecting an esophageal pressure pₑₛₒ by means of which the transpulmonary pressure Ptp can be inferred, is particularly tailored to forms of ventilation in which ventilation is carried out by means of fully automatic ventilation modes, for example, ventilation by means of closed control loops, such as those used in the Adaptive Support Ventilation (ASV ventilation) developed by the applicant. Such forms of ventilation are characterized by the fact that only minimal manual intervention by the operator is required and that the ventilation device automatically sets or adjusts important ventilation parameters such as the positive end-expiratory pressure PEEP or the maximum airway pressure Paw_max within predefined value ranges using suitable closed control loops.

The breathing gas may contain ambient air, but will typically contain a predetermined proportion of pure oxygen, hereafter referred to as FiO2, which is above the oxygen content of ambient air. The breathing gas will also typically be humidified.

The flow of the breathing gas at the airway entrance or inlet is determined using an airway inlet flow sensor 36. The airway inlet flow sensor 36 is based on detecting a pressure difference dP between an input volume 38 and an output volume 40 in communication with the input volume 38, and provides a determination of the breathing gas mass flow at the airway inlet. At the same time, the value of the airway inlet pressure Paw can be derived quite easily from the pressure signal in the output volume 40.

The pressure prevailing in the alveoli of the lungs 28, 30 is indicated by Palv in FIG. 8. This pressure depends on the airway inlet pressure Paw as well as the flow of the breathing gas V into or out of the lungs and the airway resistance R. In the case of pressure equalization between the airway inlet and the alveoli, the alveolar pressure Palv is equal to the airway inlet pressure. Such a pressure equalization has the consequence that the breathing gas flow V′ comes to a standstill. For example, a brief occlusion maneuver of the airway, i.e. airway inlet valve 18 and airway outlet valve 24 remain closed at the same time, can lead to pressure equalization. In this case, the occlusion maneuver must last just long enough for the gas flow V in the airway to stop. This is usually between 1 and 5 s. In this state, the alveolar pressure Palv can then be determined by determination of the airway inlet pressure Paw.

Both in physiological breathing and in mechanical ventilation, the flow of breathing gas is determined by a pressure difference between the alveolar pressure Palv and the airway inlet pressure Paw.

In the case of purely physiological breathing, a negative pressure difference, i.e. a negative pressure, between the alveolar pressure Palv and the airway inlet pressure Paw is generated for inhalation by expansion of the thorax (indicated at 42 in FIG. 8) and associated lowering of the pressure Ppl in the pleural space or gap 44 formed between the thorax 42 and the lungs 28, 30. Exhalation takes place passively by relaxation of the thorax and elastic resetting of the lung tissue. For this reason, during physiological breathing, the pressure in the pleural gap Ppl is always lower than the alveolar pressure Palv. The transpulmonary pressure Ptp, defined as the difference between the alveolar pressure Palv and the pressure in the pleural gap Ppl, is thus generally positive and becomes zero in the case of complete pressure equalization.

During mechanical ventilation, the breathing gas is pumped into the lungs at a positive pressure. For this reason, in mechanical ventilation, the airway inlet pressure Paw=PInsp is greater than the alveolar pressure Palv and the latter in turn is greater than the pressure in the pleural gap Ppl during the inspiration phase. It follows from these pressure relationships that the transpulmonary pressure Ppl in mechanical ventilation is positive during inspiration. During expiration, the airway inlet has an airway pressure Pexp applied thereto that is lower than the alveolar pressure Palv, so that breathing gas flows out of the alveoli. In the case of a very low airway pressure PExp, it may happen at the end of expiration, when very little gas is left in the lungs, that the pressure in the pleural gap Ppl exceeds the alveolar pressure Palv to such a high extent that part of the alveoli of the lungs collapse. The transpulmonary pressure Ptp is then negative.

Collapsing of the alveoli can be prevented by applying an additional positive pressure to the airway inlet also during the expiration phase. A positive airway pressure is then permanently applied to the airway inlet, i.e. during the inspiration phase and also during the expiration phase. This positive airway pressure is referred to as positive end-expiratory pressure or PEEP.

Consequently, the transpulmonary pressure Ptp is a suitable parameter for setting the PEEP. However, the transpulmonary pressure Ptp is not amenable to direct detection and cannot be determined from the pressures regularly detected during mechanical ventilation, as described above, either.

In FIG. 8, an additional balloon probe 46 for measuring the pressure in the esophagus 34, referred to as esophageal pressure Pₑₛₒ, is indicated in schematic manner. The balloon probe 46 is in the form of a balloon and is attached to a catheter (“esophageal catheter”) 48 inserted into the esophagus 34. The balloon probe 46 abuts internally against the wall of the esophagus 34 and provides the pressure applied to the esophagus 34 at the location of the balloon probe 46. This pressure is a good approximation of the pressure Ppl in the pleural gap when the patient is positioned appropriately. The described balloon probe 46 for detecting the esophageal pressure Pₑₛₒ and the handling of this probe is described, for example, in Benditt J., Resp. Care, 2005, 50: pp. 68-77.

Methods for in vivo calibration of a balloon probe 46 for measuring the pressure in the esophagus 34, referred to as esophageal pressure, and the use of such a balloon probe 46 in the automated ventilation of a patient are described in patent application DE 10 2021 104 993 A1 of the applicant.

The ventilation device 10 also comprises a characterization system 60 according to the invention, which enables the esophageal catheter 48 to be characterized ex vivo, i.e. before it is inserted into the patient's esophagus 34, and, if necessary, to be classified, as has been described hereinbefore with reference to FIGS. 1 to 7.

1. A method for automated ex vivo characterization of an esophageal catheter with balloon probe for determining an esophageal pressure, comprising the following steps:
filling the esophageal catheter with a measuring fluid ex vivo, in particular before placing the esophageal catheter in an esophagus;
detecting a pressure prevailing in the esophageal catheter;
determining at least one property characteristic of the esophageal catheter on the basis of the detected pressure; and
storing at least the property characteristic of the esophageal catheter.

2. The method according to claim 1,
wherein the characteristic property is detected and stored as a characteristic parameter associated with a particular pressure in the esophageal catheter.

3. The method according to claim 1,
wherein, for determining the at least one property characteristic of the esophageal catheter when detecting the pressure prevailing in the esophageal catheter, a predetermined filling quantity, i.e. a predetermined quantity of measuring fluid in the esophageal catheter, is set and the time course of the pressure is recorded or the filling quantity is changed between a start value and an end value of a measurement range for determining the characteristic property, and at each of the measurement points resulting in this way a respective pressure in the esophageal catheter detected by a pressure sensor is recorded and associated with the respective filling quantity, and wherein the characteristic property for the esophageal catheter is determined on the basis of the recorded pressures and the respectively associated filling quantity.

4. The method according to claim 3,
wherein the start value used to determine the at least one property characteristic of the esophageal catheter corresponds to a filling quantity that is greater than an upper limit of the respective measurement range for the esophageal catheter, or corresponds to a filling quantity that is less than a lower limit of the respective measurement range for the esophageal catheter; and/or
wherein a plurality of measurement values for the pressure in the esophageal catheter is determined for a respectively set predetermined filling quantity or for a respective measurement point between the start value and the end value, and wherein an average and a statistical variance for the measurement value or a variable derived therefrom are determined on the basis of the plurality of measurement values; and/or
wherein the step size between successive following measurement points is determined in adaptive manner.

5-6. (canceled)

7. The method according to claim 1,
wherein the characteristic property comprises one or more of an upper filling quantity (N_{full}) of the esophageal catheter, a filling quantity (Nₘₐₓ) of the esophageal catheter corresponding to an upper limit of a linear range, a filling quantity (Nₘᵢₙ) of the esophageal catheter corresponding to a lower limit of a linear range, a nominal filling quantity (N_{default}) of the esophageal catheter, a system volume (V_{sys}) of the esophageal catheter, a flow resistance (R_{Flow}) of the esophageal catheter and a leakage rate of the esophageal catheter, wherein the characteristic property is in particular an n-tuple whose components are n of the aforementioned properties; and/or
wherein the characteristic property comprises a leakage rate of the esophageal catheter, wherein the leakage rate of the esophageal catheter is determined in particular at a predetermined positive pressure (pₒₚ) or at a predetermined negative pressure (pᵤₚ) in the esophageal catheter.

8. (canceled)

9. The method according to claim 1,
wherein the characteristic property comprises a system volume (V_{sys}) of the esophageal catheter, wherein the system volume (V_{sys}) is the volume of the esophageal catheter when the balloon probe is fully collapsed.

10. The method according to claim 9, wherein the method comprises:
pumping measuring fluid out of the esophageal catheter until a first negative pressure (pᵤ₁) is reached in the esophageal catheter;
introducing measuring fluid into the esophageal catheter until a second negative pressure (pᵤ₂) is reached in the esophageal catheter;
associating a current second filling quantity (Nᵤ₂) of the esophageal catheter with the second negative pressure (pᵤ₂); and
determining the system volume (V_{sys}) of the esophageal catheter from the change in the filling quantity (ΔN) when the negative pressure is changed from the first negative pressure (pᵤ₁) to the second negative pressure (pᵤ₂); and/or
wherein the first negative pressure (pᵤ₁) is 15 hPa to 25 hPa, in particular 20 hPa, below the ambient pressure (p_{amb}), and wherein the second negative pressure (pᵤ₂) is 5 hPa to 15 hPa, in particular 10 hPa, below the ambient pressure (p_{amb}); and/or
wherein the method comprises:
pumping measuring fluid out of the esophageal catheter until a first negative pressure (pᵤ₁) is reached in the esophageal catheter;
introducing measuring fluid into the esophageal catheter until a second negative pressure (pᵤ₂) is reached in the esophageal catheter;
associating a current second filling quantity (Nᵤ₂) of the esophageal catheter with the second negative pressure (pᵤ₂);
introducing measuring fluid into the esophageal catheter until a third negative pressure (pᵤ₃) is reached in the esophageal catheter;
associating a current third filling quantity (Nᵤ₃) of the esophageal catheter with the third negative pressure (pᵤ₃); and
determining the system volume (V_{sys}) of the esophageal catheter from the change in the filling quantity (ΔN) when the negative pressure is changed from the second negative pressure (pᵤ₂) to the third negative pressure (pᵤ₃); and/or
wherein the first negative pressure (pᵤ₁) is 25 hPa to 35 hPa, in particular 30 hPa, below the ambient pressure (p_{amb}), wherein the second negative pressure (pᵤ₂) is 15 hPa to 25 hPa, in particular 20 hPa, below the ambient pressure (p_{amb}), and wherein the third negative pressure (pᵤ₃) is 5 hPa to 15 hPa, in particular 10 hPa, below the ambient pressure (p_{amb}).

11-13. (canceled)

14. The method according to claim 1,
wherein the characteristic property comprises a flow resistance (R_{Flow}) of the esophageal catheter, and wherein the method comprises in particular:
setting a predetermined filling level in the esophageal catheter;
changing the filling quantity in the esophageal catheter with a large time gradient, in particular by pumping or introducing measuring fluid from or into the esophageal catheter, wherein a flow rate (q) detected by a flow sensor changes strongly in terms of time, so that the pressure (pₑₛₒ) detected in the esophageal catheter also changes abruptly in terms of time;
detecting the flow rate (q) and the pressure (pₑₛₒ);
determining a temporal change (dq) of the flow rate (q) and a temporal change (dp) of the pressure (p);
determining the flow resistance (R) of the esophageal catheter from the temporal change (dq) of the flow rate (q) and the temporal change (dp) of the pressure (pₑₛₒ) or by linear regression.

15. The method according to claim 1,
wherein the characteristic property comprises an upper limit (Nₘₐₓ) and a lower limit (Nₘᵢₙ) of a linear range of the filling quantity (N) of the esophageal catheter in which the pressure (Pₑₛₒ) within the esophageal catheter changes substantially linearly with the filling quantity (N), wherein the method comprises in particular:
setting an upper filling quantity (N_{full}) of the esophageal catheter;
reducing the filling quantity (N) in the esophageal catheter stepwise or continuously while repeatedly determining the pressure (pₑₛₒ) and an associated change in the filling quantity (ΔN) in the esophageal catheter with respect to a predetermined start value (N₀) or with respect to a previous measurement point (Nⱼ);
determining, from the relationship thus determined between the pressure (p) and the change in the filling quantity (ΔN) in the esophageal catheter, the upper limit (Nₘₐₓ) and the lower limit (Nₘᵢₙ) of a linear range of the filling quantity (N) of the esophageal catheter in which the pressure (Pₑₛₒ) within the esophageal catheter changes substantially linearly with the filling quantity (N).

16. The method according to claim 14,
wherein the upper filling quantity (N_{full}) of the esophageal catheter is a filling quantity (N) at which a predetermined pressure (p_{full}), in particular a pressure of 15 hPa above the ambient pressure (p_{amb}), is measured in the esophageal catheter; and/or
wherein the filling quantity (N) in the esophageal catheter is reduced, starting from an upper filling quantity (N_{full}) in the esophageal catheter, until a predetermined lower filling quantity (N_{bot}), in particular a lower filling quantity (N_{bot}) at which a negative pressure (pₑₛₒ) of at least −5 hPa is measured in the esophageal catheter, is reached.

17. The method according to claim 1,
wherein the quantity (N) of measuring fluid in the esophageal catheter is changed stepwise between the start value and the end value and in each step a pair of measurement values is determined from the pressure (Pₑₛₒ) in the esophageal catheter and an associated change in the filling quantity (ΔN) in the esophageal catheter,
wherein stepwise changing of the filling quantity (ΔN) comprises changes with arbitrary step sizes, in particular also changes with very small step sizes and/or arbitrarily short dwell times at a respective step; and/or
wherein the change in the filling quantity (ΔN) in the esophageal catheter is determined by detecting and/or controlling the mass flow (q) of measuring fluid into the esophageal catheter and/or out of the esophageal catheter,
wherein the change in filling quantity (ΔN) is determined in particular by integrating the mass flow (q) over time (t); and/or
wherein, for determining the at least one property characteristic of the esophageal catheter, a measurement range for the esophageal catheter is determined in accordance with the detected pressure (pₑₛₒ) in the esophageal catheter;
wherein a respective characteristic property or a group of respective characteristic properties each has its own measurement range associated therewith.

18-19. (canceled)

20. The method according to claim 1,
wherein at least two measurement cycles are performed in succession, each measurement cycle comprising in particular at least the steps of filling the esophageal catheter with measuring fluid ex vivo, in particular before placing the esophageal catheter in the esophagus, and detecting a pressure (pₑₛₒ) prevailing in the esophageal catheter.

21. The method according to claim 20,
wherein a respective measurement range is associated with each of the measurement cycles and a first measurement range for the preceding measurement cycle is different from a second measurement range for the subsequent measurement cycle,
wherein the preceding measurement cycle in particular determines the measurement range for the subsequent measurement cycle; and/or
wherein the distance between successive measurement points is set differently for the preceding measurement cycle and for the subsequent measurement cycle; and/or
wherein no or at least no complete emptying of measuring fluid from the esophageal catheter takes place between a preceding measurement cycle and a subsequent measurement cycle.

22-23. (canceled)

24. The method according to claim 1,
wherein the method comprises determining a viscoelasticity of the esophageal catheter, in particular the balloon probe, wherein the method comprises in particular introducing a predetermined quantity (ΔN) of measuring fluid into the esophageal catheter and after introducing the predetermined quantity (ΔN) of measuring fluid into the esophageal catheter, determining the time required until the pressure (pₑₛₒ) in the esophageal catheter predetermined by the quantity (ΔN) of measuring fluid in the esophageal catheter has settled to a stable value.

25. The method according to claim 1,
wherein the method comprises in addition providing the esophageal catheter with at least one identifier associated with the at least one characteristic parameter stored.

26. The method according to claim 25,
wherein providing the esophageal catheter with an identifier comprises providing the esophageal catheter with a bar code or with a QR code, or attaching an RFID carrier or an NFC carrier to the esophageal catheter.

27. A characterization system for automated ex vivo characterization of an esophageal catheter with balloon probe which can be inserted into the esophagus of a patient to be ventilated, for determining an esophageal pressure, in particular for characterization before placement of the balloon probe in an esophagus, wherein the characterization system comprises:
an arrangement for filling the esophageal catheter with a measuring fluid before placing the esophageal catheter in an esophagus,
a pressure sensor for detecting a pressure (pₑₛₒ) prevailing in the esophageal catheter;
a device for determining at least one property characteristic of the esophageal catheter on the basis of the pressure (pₑₛₒ) detected in the esophageal catheter;
a storage device for storing at least the property characteristic of the esophageal catheter; and
a controller for controlling the characterization system, which is configured such that the characterization system performs a method according to claim 1.

28. The characterization system according to claim 27,
further comprising a device for providing the esophageal catheter with at least one identifier associated with the at least one characteristic property stored; and/or
further comprising a pumping device designed for introducing measuring fluid into the esophageal catheter and/or draining measuring fluid from the esophageal catheter in accordance with control commands of the at least one controller; and/or
further comprising a sensor configured to detect a mass flow (q) of measuring fluid introduced into the esophageal catheter or withdrawn from the esophageal catheter; and/or
further comprising at least one controller configured to control and/or regulate a mass flow (q) of measuring fluid introduced into the esophageal catheter or withdrawn from the esophageal catheter; and/or
comprising a leakage controller configured to control the characterization system such that it performs a method for determining a leakage rate of the esophageal catheter; and/or
comprising a system volume controller configured to control the characterization system such that it performs a method for determining a system volume (V_{sys}) of the esophageal catheter; and/or
comprising a flow resistance controller configured to control the characterization system such that it performs a method for determining a flow resistance (R_{Flow}) of the esophageal catheter; and/or
comprising a measurement range determination controller configured to control the characterization system such that it performs a method for determining an upper limit (Nₘₐₓ) and a lower limit (Nₘᵢₙ) of a linear range in which the pressure (Pₑₛₒ) within the esophageal catheter varies substantially linearly with the quantity (N) of fluid in the esophageal catheter.

29-35. (canceled)

36. A ventilation device comprising a characterization system for automated characterization of esophageal catheters with balloon probe, which can be inserted into the esophagus of a patient to be ventilated, for determining an esophageal pressure (Pₑₛₒ), in particular for preparing the esophageal catheters for use in mechanical ventilation by means of the ventilation device, according to claim 27.

37. An esophageal catheter with balloon probe for insertion into the esophagus of a patient to be ventilated for determining an esophageal pressure (Pₑₛₒ) during mechanical ventilation by means of a ventilation device, comprising:
a balloon probe which can be inserted into the esophagus of a patient to be ventilated and which can be acted upon by a measuring fluid, and
an interface for connecting a characterization system according to claim 27, wherein the interface is designed such that the characterization system is capable of determining at least one property characteristic of the esophageal catheter via the interface, the esophageal catheter in particular further comprising at least one identifier which is associated with the at least one characteristic property stored.

38. (canceled)
